(19) United States
(12) Patent Application Publication
(10) Pub. No.: US 2025/0206693 A1
(43) Pub. Date: Jun. 26, 2025

(54) TRANSPORT-MEDIATED PHOTOCATALYSTS FOR SELECTIVE PARTIAL OXIDATION OF ALKANES

(71) Applicant: The Board of Trustees of the Leland Stanford Junior University, Stanford, CA (US)

(72) Inventors: Chenlu XIE, Shanghai (CN); Arunava MAJUMDAR, Menlo Park, CA (US); Eddie SUN, Cincinnati, OH (US)

(21) Appl. No.: 18/844,107

(22) PCT Filed: Mar. 1, 2023
(86) PCT No.: PCT/US2023/063432

§ 371 Date: Sep. 5, 2024

Related U.S. Application Data
(60) Provisional application No. 63/320,721, filed on Mar. 17, 2022.

(51) Int. Cl. C07C 51/275; B01J 21/06; B01J 21/08; B01J 23/52; B01J 35/30; B01J 35/39; B01J 37/00; B01J 37/02; B01J 37/04; B01J 37/06; B01J 37/08; B01J 37/34; C07C 29/48; C07C 29/50; C07C 45/28; C07C 45/34; C07C 51/25; C07C 407/00

(52) U.S. Cl. CPC C07C 51/275; B01J 21/063; B01J 21/08; B01J 23/52; B01J 35/39; B01J 35/397; B01J 37/009; B01J 37/0211; B01J 37/0213; B01J 37/04; B01J 37/06; B01J 37/08; B01J 37/343; C07C 29/48; C07C 29/50; C07C 45/28; C07C 45/34; C07C 51/25; C07C 407/00

(57) ABSTRACT

In one aspect, the disclosure relates to a method for oxidizing alkanes to produce industrially useful solvents and other compounds. In a further aspect, the method includes the steps of contacting an alkane or mixture of alkanes with a core-shell nanoparticle and an oxidant to produce a mixture and then irradiating the mixture with UV and/or visible light. The methods are selective for desired products and do not produce overoxidized species such as, for example, carbon dioxide. In a still further aspect, the methods are scalable and can be conducted for a short time under relatively mild conditions. In an aspect, the core-shell nanoparticle includes a metal-oxide containing semiconductor core, an amorphous, radiation transparent shell, and optional metal nanoparticle dopants in the shell. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application No. 63/320,721, filed on Mar. 17, 2022, which is incorporated herein by reference in its entirety.

### STATEMENT REGARDING FEDERALLY SPONSORED RESEARCH OR DEVELOPMENT

This invention was made with Government support under contract DE-FE0031867 awarded by the Department of Energy and under contract N00014-17-1-2918 awarded by the Office of Naval Research. The Government has certain rights in the invention.

### BACKGROUND

Selective functionalization of methane to methanol and other oxygenates remains economically appealing but scientifically challenging. This is largely due to methane's inert C—H bond. In addition, the partially oxygenated products are more reactive than methane and prone to overoxidation forming CO₂ as main products. Despite enormous efforts for over a century, the direct conversion of methane to methanol is still hindered due to a trade-off between CH₄ conversion and methanol selectivity. For direct methane functionalization to replace the energy intensive industrial two-step methanol production process, new catalysts need to mitigate such trade-offs and achieve both high yield and selectivity.

From a chemistry perspective, methane can be activated either via insertion of a metal atom into the C—H bond or via hydrogen atom transfer (HAT). The former organometallic approach often requires a homogeneous metal complex catalyst, which limits its application in scalable fuel production. The latter often utilizes reactive species such as ·OH radicals to activate methane to produce methyl radicals (·CH3). The enzyme methane monooxygenase (MMO) combines HAT with an additional control of molecular transport to achieve highly selective methanol formation. Biomimicry of MMO using Fe/Cu-exchanged zeolites has also been explored, although room temperature conversion with high yields has not yet been achieved.

Photochemical methane oxidation reactions generate ·OH radicals directly from low-cost and abundant H₂O and oxygen using inorganic catalysts at room temperature and thus are promising for large scale fuel production. Previous reported photocatalysts usually consist of semiconductors such as TiO₂, ZnO, BiVO₄, and WO₃ and metal cocatalysts such as Pd, Au, Ag, and Au—Cu. However, overoxidation of methanol to CO₂ is still the limiting factor in achieving high oxygenates yields and selectivity, especially for commercialized TiO₂. On bare TiO₂, methanol is readily oxidized by holes and surface trapped ·OH radicals on the surface (FIG. 1A). Increasing the amount of water is known to increase methanol selectivity, indicating the benefits of solvation and methanol desorption from the photocatalyst surface. Hence, we hypothesize that by physically separating methanol from the TiO₂ surface, where it is least solvated and exposed to concentrated photogenerated holes and surface adsorbed ·OH, its overoxidation can be suppressed (FIG. 1B).

Despite advances in alkane oxidation research, there is still a scarcity of methods that are both effective, with high yields under room temperature conditions, and selective for specific oxidation products such as, for example, methanol and other industrially useful products without overoxidation. An ideal method would use low-cost and abundant solvents and oxidants, would be scalable, and would not require a homogeneous metal catalyst. These needs and other needs are satisfied by the present disclosure.

### SUMMARY

In accordance with the purpose(s) of the present disclosure, as embodied and broadly described herein, the disclosure, in one aspect, relates to a method for oxidizing alkanes to produce industrially useful solvents and other compounds. In a further aspect, the method includes the steps of contacting an alkane or mixture of alkanes with a core-shell nanoparticle and an oxidant to produce a mixture and then irradiating the mixture with UV and/or visible light. The methods are selective for desired products and do not produce overoxidized species such as, for example, carbon dioxide. In a still further aspect, the methods are scalable and can be conducted for a short time under relatively mild conditions. In an aspect, the core-shell nanoparticle includes a metal-oxide containing semiconductor core, an amorphous, radiation transparent shell, and optional metal nanoparticle dopants in the shell.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1D show catalyst design, preparation, and photocatalytic methane oxidation performance. (FIG. 1A) Schematic illustration of methanol overoxidation on bare TiO₂ catalysts by photogenerated holes and hydroxyl radicals on the surface. (FIG. 1B) Schematic illustration of the SiO₂ coating layer on TiO₂ to prevent methanol overoxidation. (FIG. 1C) HR-TEM image of AuPd/TiO₂ catalysts. (FIG. 1D) HR-TEM image of TiO₂@SiO₂—AuPd catalysts.

FIGS. 2A-2D show catalytic performance of photocatalytic methane oxidation. (FIG. 2A) Photocatalytic oxidation of methane over different TiO₂ catalysts. Reaction conditions: 10 mg of catalysts, 100 mL of H₂O, 6.9 bar of CH₄, 2.76 bar of O₂, 1 h reaction time, reaction temperature of 25±3° C., light source of 365 nm UV LED, 130 mW/cm². Error bars represent standard deviations obtained from three independent measurements. (FIG. 2B) Effect of water amount on the catalytic performance of TiO₂@SiO₂—AuPd and AuPd/TiO₂. Reaction conditions: 10 mg of catalysts, 6.90 bar of CH₄, 2.75 bar of O₂, 1 h reaction time, 25±3° C. reaction temperature, light source of 365 nm UV LED, light intensity of 130 mW/cm². (FIG. 2C) Product yields and oxygenates selectivity over TiO₂@SiO₂—AuPd with different SiO₂ thickness. Reaction conditions: 10 mg of TiO₂@SiO₂—AuPd, 100 mL of H₂O, 6.9 bar of CH₄, 2.75 bar of O₂, 1 h, 25±3° C., light source of 365 nm UV LED, 130 mW/cm². (FIG. 2D) Productivity assessment for oxygenates obtained at different light intensities. Reaction conditions: 10 mg of TiO₂@SiO₂—AuPd catalysts, 100 mL of H₂O, 6.9 bar of CH₄, 2.75 bar of O₂, 1 h, 25±3° C. Oxygenates product selectivity (%)=moles of (CH₃OOH+CH₃OH+HCHO+HCOOH)×100%/total moles of products.

FIGS. 3A-3C show investigation of the role of SiO₂ shell and AuPd nanoparticles. (FIG. 3A) Comparison of catalytic activity of TiO₂@SiO₂—AuPd and its individual components (i.e., SiO₂, AuPd/SiO₂, and TiO₂@SiO₂) and the physical mixture. (FIG. 3B) Product yields and oxygenates selectivity on TiO₂@SiO₂—AuPd with different TiO₂@SiO₂ annealing temperature. Reaction condition: 10 mg of photo-catalyst, 100 mL of H₂O, 6.9 bar of CH₄, 0.28 bar of O₂, 2.47 bar of Ar, 1 h reaction time, 25±3° C. reaction temperature, light source of 365 nm UV LED with intensity of 130 mW/cm². (FIG. 3C) O₂ partial pressure dependence on TiO₂@SiO₂—AuPd with different SiO₂ thickness. Reaction condition in (FIG. 3A) and (FIG. 3C): 10 mg of photocatalyst, 100 mL of H₂O, 6.9 bar of CH₄, 2.75 bar of O₂, 1 h reaction time, reaction temperature of 25±3° C., light source of 365 nm UV LED, 130 mW/cm².

FIGS. 4A-4E show a proposed reaction mechanism. (FIG. 4A) Schematic of the proposed reaction mechanism for photocatalytic CH₄ oxidation: CB, conduction band; VB, valence band. (FIG. 4B) UV-vis absorption spectra of XTT-formazan product for O₂·⁻ detection over different photocatalysts under UV irradiation. (FIG. 4C) Fluorescence spectra of the produced 7-hydroxycoumarin for ·OH radical detection over different photocatalysts under UV irradiation. (FIG. 4D) Schematic of the permeation behavior of key species on TiO₂@SiO₂ and (FIG. 4E) TiO₂@SiO₂—AuPd catalysts.

FIGS. 5A-5B show generalization of the photocatalyst design strategy. (FIG. 5A) Product yields and oxygenates selectivity of photocatalytic ethane oxidation. Reaction conditions: 10 mg of photocatalyst, 100 mL of H₂O, 6.9 bar of C₂H₆, 2.75 bar of O₂, 25±3° C. reaction temperature, light source of 365 nm UV LED, 130 mW/cm². (FIG. 5B) H₂O₂ used as ·OH radical source on noble-metal-free photocatalysts. Reaction conditions: 10 mg of photocatalyst, 20 mL of H₂O, 2 mL of 50 mM H₂O₂, 6.9 bar of CH₄, 0.07 bar of O₂, 25±3° C. reaction temperature, 1 h reaction time, light source of 365 nm UV LED, 130 mW/cm².

FIG. 6 shows a synthesis procedure for TiO₂@SiO₂—AuPd.

FIG. 7 shows transmission electron microscopy (TEM) images of AuPd/TiO₂ (AuPd loading is 1 wt %).

FIGS. 8A-8D show TEM and HR-TEM images of TiO₂@SiO₂—AuPd (AuPd loading is 1 wt %). AuPd nanoparticles have an average diameter of 4 nm and a lattice spacing of 0.23 nm from HR-TEM images, which is different than the typical values for metallic gold and palladium. This confirms the formation of alloyed particles rather than separated Au or Pd phases.

FIGS. 9A-9B show transmission electron microscopy (TEM) and high-resolution (HR)-TEM images of TiO₂@SiO₂.

FIGS. 10A-10F show HAADF-STEM (FIG. 10A) images and elemental mapping of (FIG. 10B) TiO₂@SiO₂—AuPd with energy dispersive X-ray spectroscopy (EDS). Corresponding EDS elemental mapping for (FIG. 10C) Ti, (FIG. 10D) Si, (FIG. 10E) Au, (FIG. 10F) Pd, respectively. Scale bar: 70 nm.

FIGS. 11A-11B show the XRD pattern and N₂ adsorption isotherm of the photocatalysts. (FIG. 11A) The XRD patterns of TiO₂, TiO₂@SiO₂, TiO₂@SiO₂—AuPd are identical, indicating the amorphous nature of the SiO₂ shell. No peak from AuPd was observed due to the low loading amount (1 wt %) of AuPd. (FIG. 11B) N₂ adsorption isotherm of TiO₂ and TiO₂@SiO₂ with a 5 nm SiO₂ shell. The surface areas of TiO₂ and TiO₂@SiO₂ measured from by Brunauer-Emmett-Teller (BET) method are 60.1 m²/g and 89.9 m²/g, respectively. Since the N₂ absorption curve of TiO₂@SiO₂ largely follows a type Ill curve, it is speculated that there are no or only a very small number of micropores. The higher surface area of TiO₂@SiO₂ compared to bare TiO₂ is largely due to the increase of the external surface area, which is believed to come from the surface roughness of SiO₂ (amorphous nature).

FIGS. 12A-12E show X-ray absorption near edge structure (XANES) spectra of TiO₂@SiO₂—AuPd at Au L₃-edge (FIG. 12A) and Pd K-edge (FIG. 12B) and corresponding extended X-ray absorption fine structure (EXAFS) spectra of Au L₃-edge and its best fitting (FIG. 12C) and Pd K-edge (FIG. 12D). (FIG. 12E) k-space EXAFS data spectrum and the best fitting of TiO₂@SiO₂—AuPd at Au L₃ edge. Note: Both Au and Pd are oxidized in the TiO₂@SiO₂—AuPd catalysts. For Pd, it is a mixed phase of AuPd alloy and PdO. Au is slightly oxidized as evidenced by the Au—O bond after annealing. XANES of AuPd—SiO₂ shows the increased white line intensity, which indicates Au oxidation. A subtle Au—O bond is also observed, as shown by the EXAFS fitting results (Table 4).

FIG. 13 shows UV-vis diffuse reflectance spectra (UV-DRS) of TiO₂, TiO₂@SiO₂ and TiO₂@SiO₂—AuPd.

FIGS. 14A-14B show a schematic (FIG. 14A) and image (FIG. 14B) of the custom photocatalytic batch reactor setup used in the disclosed process.

FIGS. 15A-15B show ¹³CH₄ and ¹⁸O₂ isotope labeling experiments on TiO₂@SiO₂—AuPd. (FIG. 15A) ¹H NMR spectrum of methane oxidation reaction on TiO₂@SiO₂—AuPd carried out with ¹³CH₄ and ¹²CH₄ mixture (25% ¹³CH₄ and 75% ¹²CH₄). DMSO is used as internal standard. ¹³CH₃OH (δ=3.46 and δ=3.22), ¹³CH₃OOH (δ=3.97 and δ=3.73), and H¹³COOH (δ=8.54 and δ=8.20) satellite peaks are observed along with main ¹²C peaks (δ=3.34, δ=3.85 and δ=8.37, respectively). The integrated area of the satellite peaks is about one-third that of main ¹²C peak corresponding to the 1:3 ratio of ¹³CH₄ and ¹²CH₄. (FIG. 15B) Gas chromatography-mass spectrometry (GC-MS) spectra of CH₃OH formed during photocatalytic methane oxidation with TiO₂@SiO₂—AuPd via ¹⁸O2+H₂¹⁶O. m/z, mass/charge ratio. The results show that more than 99% of methanol contains ¹⁸O instead of ¹⁶O, indicating that O₂ is the oxygen source instead of water to form methanol from methane.

FIGS. 16A-16D show the effect of SiO₂ thickness on the disclosed system. TEM images TiO₂@SiO₂—AuPd with different SiO₂ thickness: (FIGS. 16A-16B) 17.5 nm; (FIGS. 16C-16D), 1.5 nm.

FIGS. 17A-17B show (FIG. 17A) time course evolution of product yields, oxygenates selectivity, and CH₄ conversion. Reaction conditions: 10 mg TiO₂@SiO₂—AuPd, 100 mL H₂O, 6.9 bar CH₄, 2.75 bar O₂, 25±3° C., light source: 365 nm UV LED, 130 mW/cm². (FIG. 17B) Product yields, oxygenates selectivity and CH₄ conversion at different CH₄/O₂ ratios. Reaction conditions: 10 mg TiO₂@SiO₂—AuPd, 100 mL water, total pressure 9.65 bar, 25±3° C. reaction temperature, reaction time: 3 h, light source: 365 nm UV LED with intensity of 130 mW/cm².

FIGS. 18A-18B show productivity assessment for oxygenates on AuPd/TiO₂ and TiO₂@SiO₂ obtained under (FIG. 18A) 130 mW/cm² and (FIG. 186) 470 mW/cm². Reaction condition: 10 mg photocatalyst, 100 mL H₂O, 6.90 bar CH₄, 2.75 bar O₂, 1 h reaction time, 25±3° C. reaction temperature, light source: 365 nm UV LED. Note: The total product yields of AuPd/TiO₂ increased from 14.7 mmol/gcat·h to 38.7 mmol/gcat·h (by 2.6 times) after increasing the light intensity from 130 mW/cm² to 470 mW/cm², while for TiO₂@SiO₂—AuPd, the total yields only increased 1.6 times (from 16.3 mmol/gcat·h to 26.8 mmol/gcat·h).

FIGS. 19A-19C show (FIG. 19A) cycling tests of photocatalytic oxidation of CH₄. Reaction condition: 10 mg TiO₂@SiO₂—AuPd, 100 mL H₂O, 6.9 bar CH₄, 2.75 bar O₂, 1 h, 25±3° C., light source: 365 nm UV LED, 130 mW/cm². (FIGS. 19B-19C) TEM images of TiO₂@SiO₂—AuPd after 5 cycles of reactions. No obvious morphology changes were observed.

FIG. 20 shows a TEM image of AuPd/SiO₂.

FIG. 21 shows N₂ adsorption isotherm of TiO₂@SiO₂ annealed at 550° C. for 4 h in air. The surface areas measured from by Brunauer-Emmett-Teller (BET) method is 59.6 m²/g, which is lower than that of TiO₂@SiO₂—AuPd annealed at 350° C. for 2 h. This indicates that the TiO₂@SiO₂ (550° C., 4 h) has denser structure and would lead to higher restriction on the oxygen species transport, which is reflected by the lower product yields.

FIGS. 22A-22D show product yields and oxygenate selectivity over (FIG. 22A) TiO₂@SiO₂—AuPd with 5 nm SiO₂ shell, (FIG. 22B) AuPd/TiO₂, (FIG. 22C) TiO₂@SiO₂—AuPd with 1.5 nm SiO₂ shell, and (FIG. 22D) TiO₂@SiO₂—AuPd with 17.5 nm SiO₂ shell thickness under different O₂ pressures. Reaction conditions: 10 mg photocatalyst, 100 mL H₂O, 6.9 bar CH₄, 1 h reaction time, reaction temperature: 25±3° C., light source: 365 nm UV LED, 130 mW/cm².

FIGS. 23A-23C show photogenerated radical measurements. (FIG. 23A) XTT dissolved in aqueous solution was used as the probe to capture O₂·⁻ radicals to give a formazan product with absorption at 470 nm. (FIG. 23B) Coumarin molecules were used as probe to capture ·OH radicals and produce fluorescent 7-hydroxycoumarin (7-HC) that can be quantified by photoluminescence measurement. (FIG. 23C) 7-HC Fluorescence intensity vs. wavelength of emission light of various concentration of 7-HC. Inserted: Fluorescence calibration curve.

FIGS. 24A-24C show SEM-EDS spectrum of TiO₂@SiO₂—AuPd.

FIG. 25 shows a proposed reaction mechanism based on hydrogen spillover for photocatalytic methane oxidation on TiO₂@SiO₂—AuPd (denoted as “H transport mechanism”). Note: According to the analysis in the Examples combined with a series of experiments including O₂ partial pressure tuning and silica thickness control, it is speculated that this H transport mechanism is not well supported by the experimental results.

FIG. 26 shows catalytic performance of TiO₂@SiO₂—AuPd with AuPd loading amount. Reaction condition: 10 mg photocatalyst, 100 mL H₂O, 6.90 bar CH₄, 2.75 bar O₂, 1 h reaction time, 25±3° C. reaction temperature, light source: 365 nm UV LED, 130 mW/cm². Note: 1 wt % AuPd loaded photocatalyst produced the optimum oxygenates yields. Further increasing AuPd loading led to lower oxygenates production and can be attributed to the shielding effect: AuPd blocks light from reaching the TiO₂ surface.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The high activation barrier of the C—H bond in methane, combined with the high propensity of methanol and other liquid oxygenates toward overoxidation to CO₂, have historically posed significant scientific and industrial challenges to the selective and direct conversion of methane to energy-dense fuels and chemical feedstocks. Herein is disclosed a unique core-shell nanostructured photocatalyst, silica encapsulated TiO₂ decorated with AuPd nanoparticles (TiO₂@SiO₂—AuPd), for alkane oxidation with high yields and high selectivity. In one aspect, the core-shell catalytic particles include a metal oxide core optionally coated with a nanoscopic shell that selectively prevents methanol overoxidation on its surface and possesses high selectivity and yield of oxygenates even at high UV intensity, without greatly hindering alkane conversion. This transport selective architecture is composed of an amorphous layer, which can contain SiO₂, and can be decorated with metal nanoparticles such as, for example, AuPd nanoparticles (SiO₂—AuPd). While it is noted that AuPd nanoparticles have a well-known role in methanol formation, without wishing to be bound by theory, the metallic nanoparticle decorations serve a second role in this photocatalytic architecture by allowing for the diffusion of species necessary for methane and/or other alkane oxidation.

In one aspect, disclosed herein is a method for oxidizing an alkane, the method including at least the steps of contacting a composition including the alkane with a core-shell nanoparticle and an oxidant to produce a mixture and irradiating the mixture to produce one or more oxidized alkane species. In a further aspect, the alkane can be a C1-C6 linear, branched, or cyclic alkane, or can be a mixture of different C1-C6 linear, branched, or cyclic alkanes. In some aspects, the alkane can be methane or ethane, although other alkanes are also contemplated and should be considered disclosed. Further in this aspect, the C—H activation mechanism is very similar among all alkanes. In an aspect, the composition can include at least 5 vol % of the alkane, or from about 20 to about 100 vol % of the alkane, optionally from about 20 to about 50 vol % of the alkane, from about 40 to about 60 vol % of the alkane, or from about 50 to about 100 vol % of the alkane. In an alternative aspect, the method can be carried out with an alkane partial pressure of from about 0.1 to about 200 bar, from about 6 to about 200 bar, from about 1 to about 150 bar, or from about 6 to about 30 bar.

In one aspect, the oxidant can be O₂, H₂O₂, N₂O, or a combination thereof. In some aspects, when the oxidant is O₂, the O₂ partial pressure can be expressed in terms of ratio of alkane (e.g. CH₄ or another alkane) partial pressure to O₂ partial pressure. In an aspect, the alkane to O₂ ratio can be about 100:0.5, or about 100:1, or about 2:1.

In any of these aspects, the mixture can further include a solvent, such as, for example, water. The solvent can be present in a bench-scale reaction in an amount of from about 1 to about 1000 mL, from about 20 mL to about 500 mL, from about 75 to about 150 mL, or from about 75 to about 100 mL, about 100 to about 125 mL, or from about 125 to about 150 mL. In some aspects, when the reaction is scaled up, for every 1 gram of catalyst (i.e. core-shell nanoparticles) used, from about 2 L to about 100 L of solvent can be used, or from about 2 L to about 50 L of solvent can be used.

In an aspect, a core of the core-shell nanoparticle includes at least one semiconductor, including, but not limited to TiO₂, SrTiO₃, ZnO, BiVO₄, In₂O₃, carbon nitride, and combinations thereof. Oxide-containing semiconductors and other semiconductors having a band gap of from about 2 to about 4 eV not listed herein are also contemplated and should be considered disclosed. Without wishing to be bound by theory, any oxide semiconductor generating holes that can react with water to form OH radicals and/or electrons can be useful as part or all of the composition of the core.

In a further aspect, a shell of the core-shell nanoparticle includes at least one oxide transparent to UV or visible radiation. Further in this aspect, the shell may be amorphous. In still another aspect, the shell can be hydrophilic. In some aspects, the at least one oxide is or includes SiO₂. In a further aspect, any hydrophilic, amorphous, and UV or visible light transparent oxide is contemplated for the disclosed shells. In another aspect, the shell has a thickness of from about 0.5 nm to about 20 nm, or from about 0.5 nm to about 10 nm, or from about 1 nm to about 8 nm. In some aspects, the shell thickness is about 5 nm. In another aspect, the thickness of the shell layer correlates with the type of oxide in the shell as well as its pore size and structure.

In some aspects, the shell further includes a dopant or “decoration” such as, for example, gold, platinum, palladium, copper, ruthenium, rhenium, or any combination thereof, including, but not limited to, combinations such as AuPd and CuPd. In one aspect, the dopant can be present in an amount of from about 0.1 to about 20 wt %, or from about 0.1 to about 10 wt %, from about 1 to about 5 wt %, or from about 2 to about 8 wt % relative to the weight of the nanoparticles. Without wishing to be bound by theory, having a metal nanoparticle or decoration loading above about 50% may block light absorption and interfere with the disclosed reactions. In some aspects, in the disclosed methods, the core-shell nanoparticles can be present in an amount of at least about 5 mg, or of at least about 10 mg. In one aspect, when the oxidant is O₂, any metal nanoparticle that can dissociate O₂ can be used for the oxidant.

In one aspect, an exemplary nanoparticle can include a TiO₂ core, an SiO₂ shell, and a dopant consisting of a combination of gold and palladium. Further in this aspect, the oxidant can be O₂. Another exemplary nanoparticle can include a TiO₂ core, an SiO₂ shell, and no dopant. Further in this aspect, the oxidant can be H₂O₂.

In an aspect, the alkane can be methane and the one or more oxidized alkane species can be formic acid, formaldehyde, methanol, methyl hydroperoxide, carbon dioxide, or any combination thereof. Further in this aspect, the amount of methanol produced is at least 2, 4, 6, or 8 times greater than the amount of carbon dioxide produced. Still further in this aspect, the amount of carbon dioxide produced can be less than 2 mmol per grams of catalyst (i.e., core-shell nanoparticle) per hour relative to a total amount of oxidized alkanes produced. In a still further aspect, the alkane can be ethane and the one or more oxidized alkane species can be acetic acid, acetaldehyde, ethanol, or any combination thereof.

In one aspect, irradiation can be accomplished with UV or visible light having a wavelength of from about 320 to about 780 nm. In another aspect, irradiation can be accomplished using a xenon lamp or daylight. In still another aspect, irradiation can be accomplished using UV light having a wavelength of about 365 nm. In another aspect, the light can have a flux of greater than about 10 mW/cm², of from about 10 to about 1000 mW/cm², of from about 100 to about 500 mW\cm², of from about 500 to about 1000 mW/cm², of from about 130 to about 470 mW/cm², or of about 130 to 200 mW/cm², about 200 to about 350 mW/cm², or about 350 to about 470 mW/cm².

In one aspect, the method can be carried out under mild conditions for a short time period and is scalable. In some aspects, the method can be carried out as a batch process or as a continuous process. In a further aspect, the method can be carried out from about 0 to about 70° C., from about 5 to about 70° C., from about 15 to about 70° C., from about 15 to about 45° C., from about 45 to about 70° C., from about 22 to about 28° C., from about 22 to about 25° C., or from about 25 to about 28° C. In some aspects, the method can be carried out as a continuous process for from about 10 minutes to about 24 hours, or from about 15 minutes to about 6 hours, or for about an hour.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a catalyst,” “an oxidant,” or “an alkane,” includes, but is not limited to, mixtures or combinations of two or more such catalysts, oxidants, or alkanes, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less’ and should be interpreted to include the specific ranges of ‘about x,’ ‘about y’, and ‘about z’ as well as the ranges of ‘less than x’, less than y’, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a catalyst refers to an amount that is sufficient to achieve the desired improvement in the property modulated by the formulation component, e.g. achieving the desired level of production of methanol relative to the amount of methane originally present in the reaction mixture. The specific level in terms of wt % or vol % in a composition required as an effective amount will depend upon a variety of factors including the amount and type of alkane to be converted, amount and type of metal particles decorating the shell of the core-shell particle, wavelength and photon flux of irradiation to which the reaction mixture is exposed, and desired end products.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered Aspects, which should not be confused with the claims.

Aspect 1. A method for oxidizing an alkane, the method comprising:
(a) contacting a composition comprising the alkane with a core-shell nanoparticle and an oxidant to produce a mixture; and
(b) irradiating the mixture to produce one or more oxidized alkane species.

Aspect 2. The method of aspect 1, wherein the alkane comprises a C1-C6 linear, branched, or cyclic alkane.

Aspect 3. The method of aspect 1, wherein the alkane comprises methane or ethane.

Aspect 4. The method of aspect 1, wherein the composition comprises at least about 5 vol % of the alkane.

Aspect 5. The method of aspect 4, wherein the composition comprises from about 20 to about 100 vol % of the alkane.

Aspect 6. The method of aspect 1, wherein the method is conducted with an alkane partial pressure of from about 0.1 to about 200 bar.

Aspect 7. The method of aspect 1, wherein the oxidant comprises O₂, H₂O₂, N₂O, or any combination thereof.

Aspect 8. The method of aspect 7, wherein the method is conducted with a ratio of alkane partial pressure to O₂ partial pressure of from 100:0.5 to about 2:1.

Aspect 9. The method of aspect 1, wherein the mixture further comprises a solvent.

Aspect 10. The method of aspect 9, wherein the solvent comprises water.

Aspect 11. The method of aspect 9, wherein the solvent is present at from about 2 L to about 100 L of solvent per gram of core-shell nanoparticles.

Aspect 12. The method of aspect 1, wherein a core of the core-shell nanoparticle comprises at least one semiconductor.

Aspect 13. The method of aspect 12, wherein the semiconductor comprises an oxide with a band gap of from about 2 to about 4 eV.

Aspect 14. The method of aspect 12, wherein the at least one semiconductor comprises TiO₂, SrTiO₃, ZnO, BiVO₄, In₂O₃, carbon nitride, or any combination thereof.

Aspect 15. The method of aspect 1, wherein a shell of the core-shell nanoparticle comprises at least one oxide transparent to UV or visible radiation.

Aspect 16. The method of aspect 15, wherein the at least one oxide comprises SiO₂.

Aspect 17. The method of aspect 15, wherein the shell has a thickness of from about 0.5 nm to about 20 nm.

Aspect 18. The method of aspect 16, wherein the thickness is about 5 nm.

Aspect 19. The method of aspect 1, wherein a shell of the core-shell nanoparticle further comprises a dopant.

Aspect 20. The method of aspect 19, wherein the dopant comprises gold, platinum, palladium, copper, rhenium, ruthenium, or any combination thereof.

Aspect 21. The method of aspect 19, wherein the dopant is present in an amount of from about 0.1 wt % to about 10 wt % relative to the total weight of the nanoparticles.

Aspect 22. The method of aspect 21, wherein the dopant is present at about 5 wt % relative to the total weight of the nanoparticles.

Aspect 23. The method of aspect 1, wherein the core-shell nanoparticles are present in an amount of about 5 mg.

Aspect 24. The method of aspect 1, wherein the mixture is irradiated using light.

Aspect 25. The method of aspect 1, wherein the light has a wavelength from about 320 nm to about 780 nm.

Aspect 26. The method of aspect 24, wherein the mixture is irradiated using 365 nm UV light.

Aspect 27. The method of aspect 24, wherein the light has a flux greater than about 10 mW/cm².

Aspect 28. The method of aspect 27, wherein the light has a flux of from about 130 to about 470 mW/cm².

Aspect 29. The method of aspect 1, wherein the method is carried out at a temperature of from about 0 to about 70° C.

Aspect 30. The method of aspect 1, wherein the method is carried out as a batch process or a continuous process.

Aspect 31. The method of aspect 30, wherein the method is carried out as a continuous process for from about 10 minutes to about a 24 hours.

Aspect 32. The method of aspect 19, wherein a core of the core-shell nanoparticle comprises TiO₂, wherein the shell of the core-shell nanoparticle comprises SiO₂, and wherein the dopant comprises gold and palladium.

Aspect 33. The method of aspect 19, wherein the oxidant comprises O₂.

Aspect 34. The method of aspect 1, wherein a core of the core-shell nanoparticle comprises TiO₂, wherein a shell of the core-shell nanoparticle comprises SiO₂, and wherein the oxidant comprises H₂O₂.

Aspect 35. The method of aspect 1, wherein the alkane comprises methane and the one or more oxidized alkane species comprises formic acid, formaldehyde, methanol, methyl hydroperoxide, carbon dioxide, or any combination thereof.

Aspect 36. The method of aspect 35, wherein an amount of methanol produced is at least 4 times greater than an amount of carbon dioxide produced.

Aspect 37. The method of aspect 35, wherein an amount of carbon dioxide produced is less than 2 mmol per grams of core-shell nanoparticle per hour relative to a total amount of oxidized alkane species produced.

Aspect 38. The method of aspect 1, wherein the alkane comprises ethane and the one or more oxidized alkane species comprises acetic acid, acetaldehyde, ethanol, or any combination thereof.

Aspect 39. An oxidized alkane produced by the method of aspect 1.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Process Overview

Herein, a core-shell type photocatalytic architecture for methane oxidation with high yields and selectivity has been developed. In this architecture, the TiO₂ core is coated with a nanoscopic shell that selectively blocks methanol without greatly hindering methane conversion. This transport selective architecture is composed of an amorphous SiO₂ layer with decorated AuPd nanoparticles (SiO₂—AuPd). While the intended purpose of the incorporation of AuPd nanoparticles in the design was because of its well-known role in methanol formation, it serves a second role in this photocatalytic architecture by allowing for the diffusion of species necessary for methane oxidation. Under a UV flux of 130 mW/cm², TiO₂@SiO₂—AuPd produced 15.4 mmol/gcat·h of liquid oxygenates with 94.5% selectivity at 9.65 bar total pressure of CH₄ and O₂. At this reaction condition, its SiO₂-free counterparts (AuPd/TiO₂) produced CO₂ as the major product. Due to the protective silica layer, the high oxygenates selectivity can be maintained at various reaction conditions, which enables the use of higher UV flux (470 mW/cm²) to produce 21.3 mmol/gcat·h of oxygenates with 80% selectivity. The working principle of the catalyst was further elucidated by a series of systematic studies varying the catalyst structure and reaction conditions. It is also shown that this core-shell catalyst design is generalizable for selective oxidation of other alkanes.

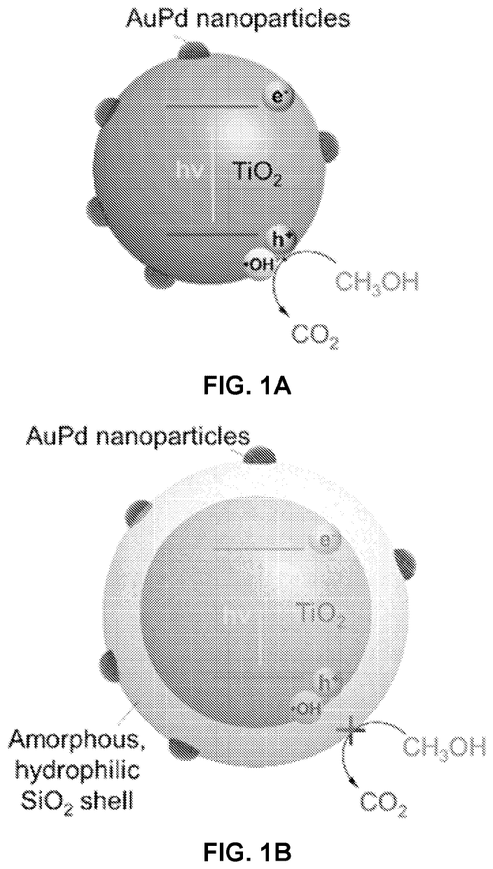

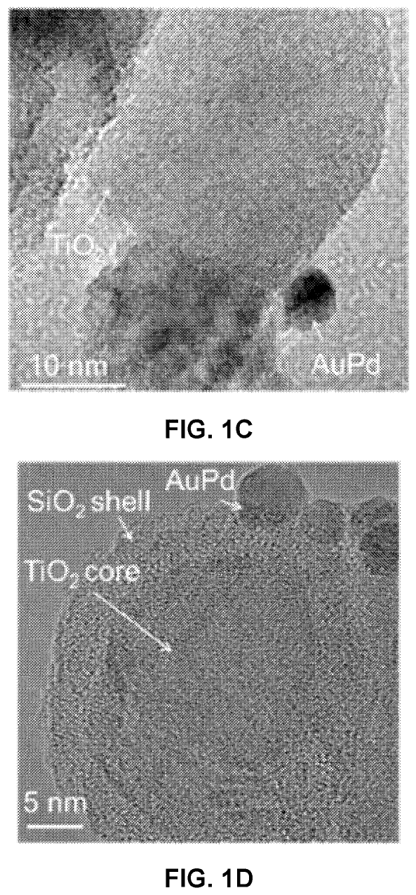

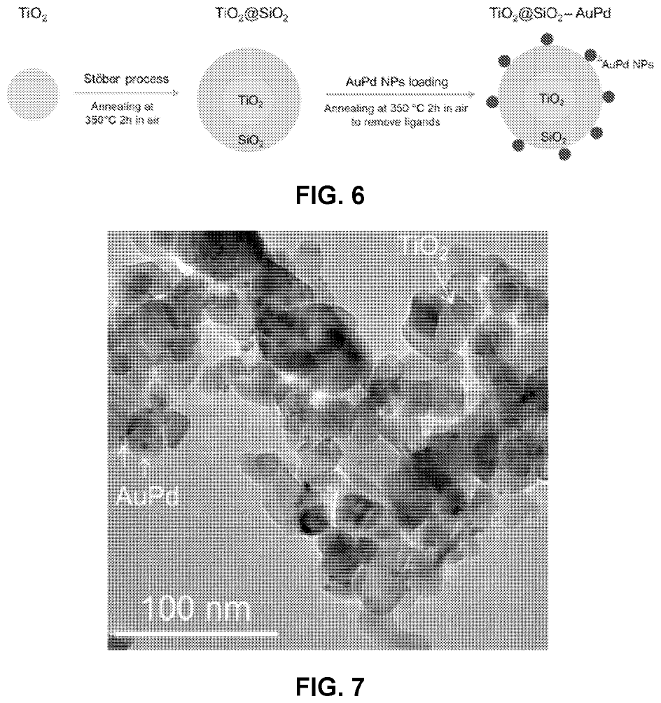

The silica shell was prepared using a modified Stöber method on P25 TiO₂ (denoted as TiO₂@SiO₂) with tunable thickness. AuPd colloids were loaded onto TiO₂@SiO₂ (denoted as TiO₂@SiO₂—AuPd), followed by calcination in air at 350° C. (FIG. 6). The total metal loading was around 1 wt % (ICP-OES) (Table 1). For comparison, AuPd/TiO₂ was prepared using the same method (FIGS. 1C and 7).

TABLE 1
ICP-OES Results for Different Photocatalysts

| | Metal mass loading (%) | |
|---|---|---|
| Catalyst | (Au, Pd in total) | Au/Pd molar ratio |
| AuPd/TiO₂ | 1.14 | 1.28 |
| TiO₂@SiO₂—AuPd | 1.01 | 1.31 |
| AuPd/SiO₂ | 0.89 | 1.33 |

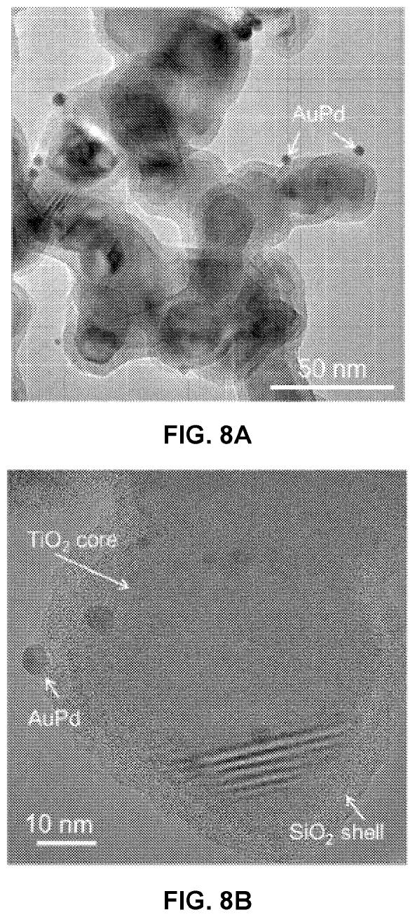

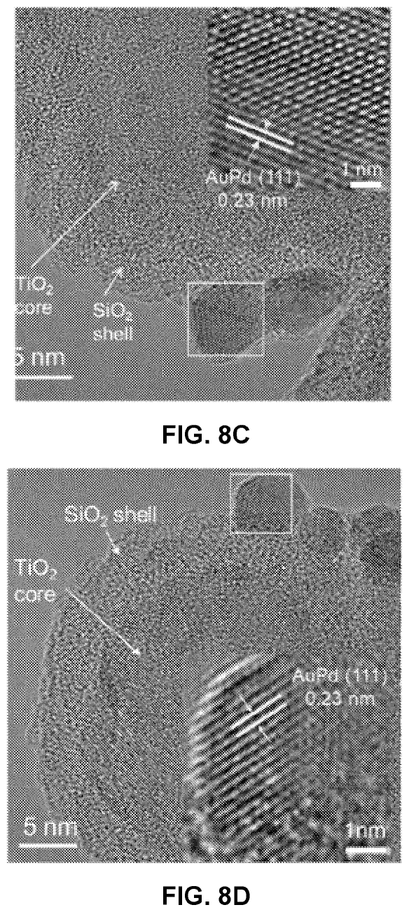

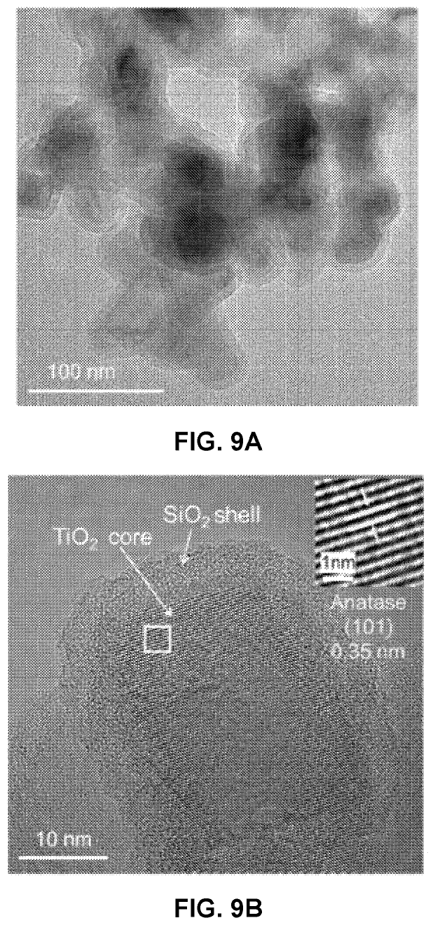

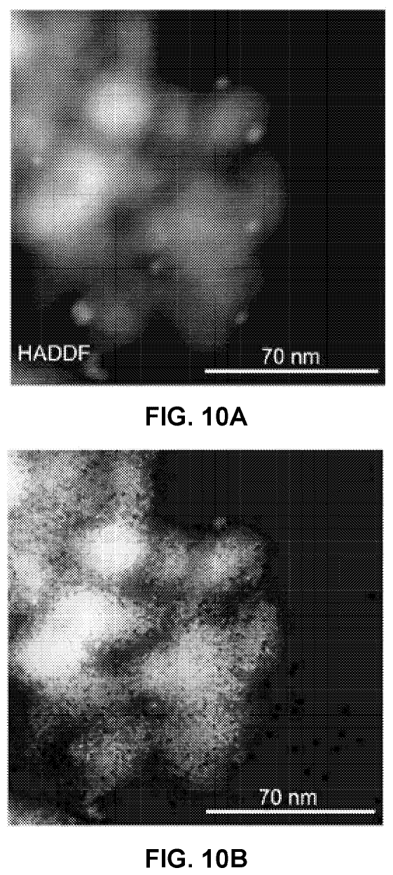

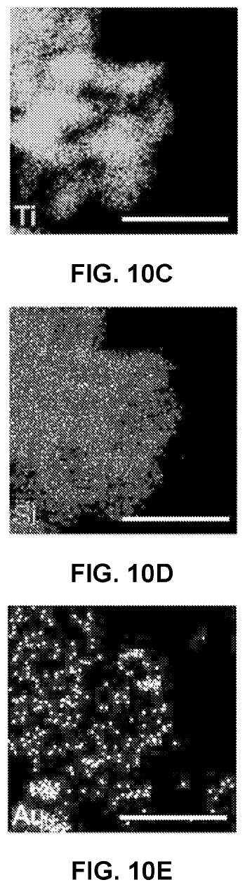

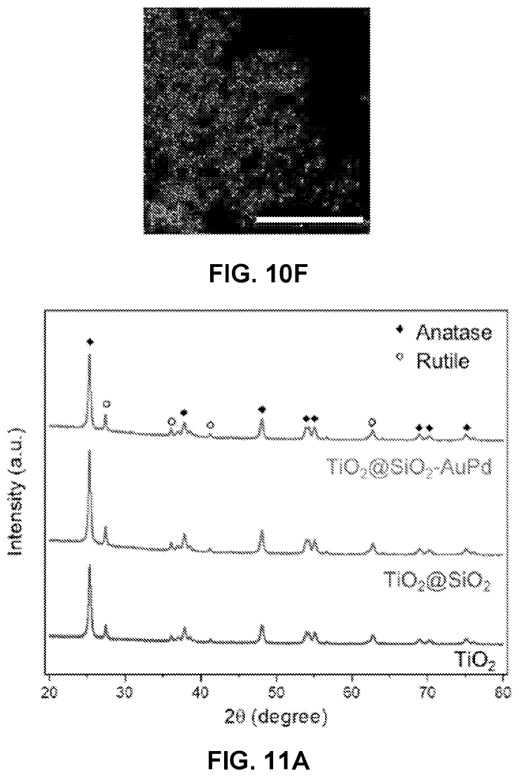

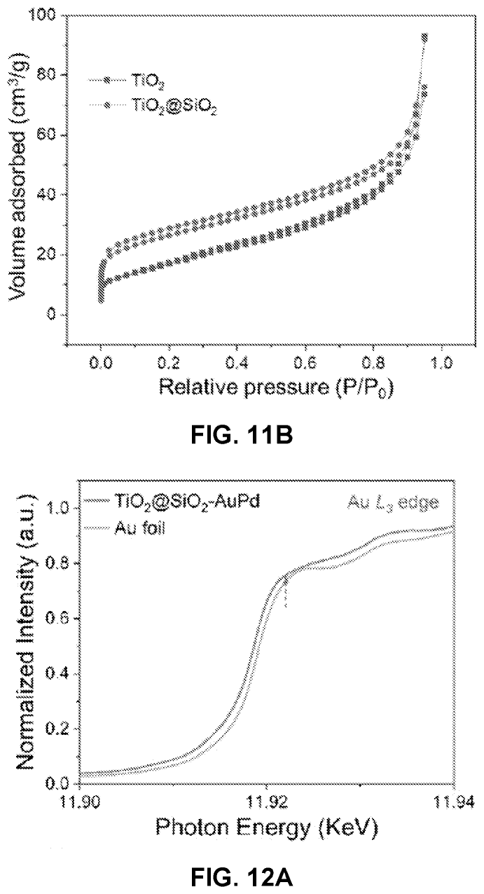

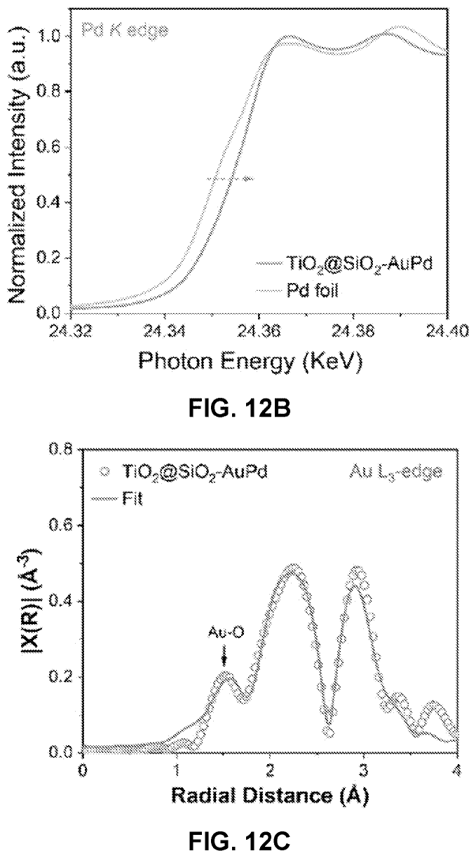

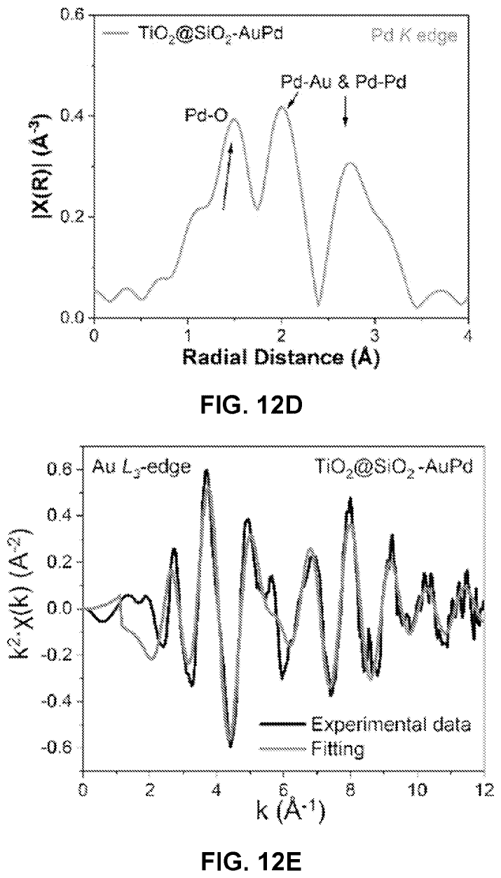

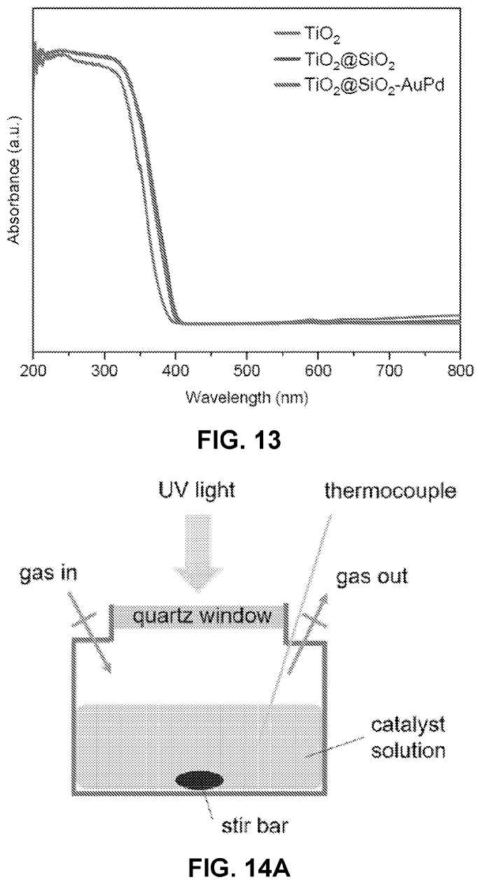

Transmission electron microscopy (TEM), energy-dispersive X-ray spectroscopy (EDS), and X-ray diffraction confirmed that the TiO₂ nanoparticles were fully encapsulated by a uniform amorphous SiO₂ shell with about 5 nm thickness (FIGS. 1D and 8A-11B). The surface areas of TiO₂ and TiO₂@SiO₂ were determined by N₂ adsorption to be 60.1 and 89.9 m²/g, respectively (FIG. 11B), indicating the SiO₂ shell has no or only small amount of micropores. The formation of alloyed AuPd particles was confirmed by TEM and EDS (FIGS. 8A-8D and 10A-10F). Both Au and Pd of TiO₂@SiO₂—AuPd were in an oxidized state relative to their metallic states, as evidenced by X-ray absorption near edge structure (XANES) and extended X-ray absorption fine structure (EXAFS) studies (FIGS. 12A-12E). The UV-diffusive reflective spectra (UV-DRS) of TiO₂ and TiO₂@SiO₂ are almost identical (FIG. 13), suggesting that the SiO₂ coating does not significantly affect light absorption.

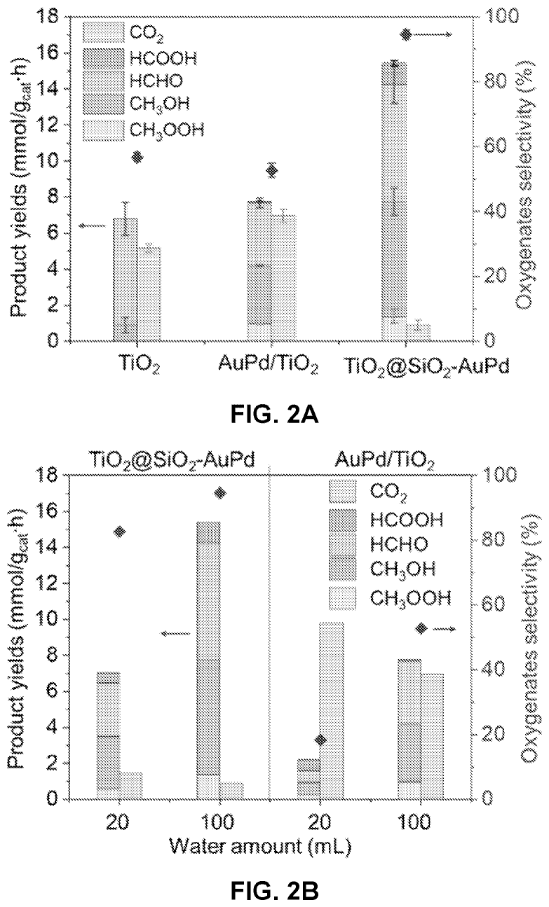

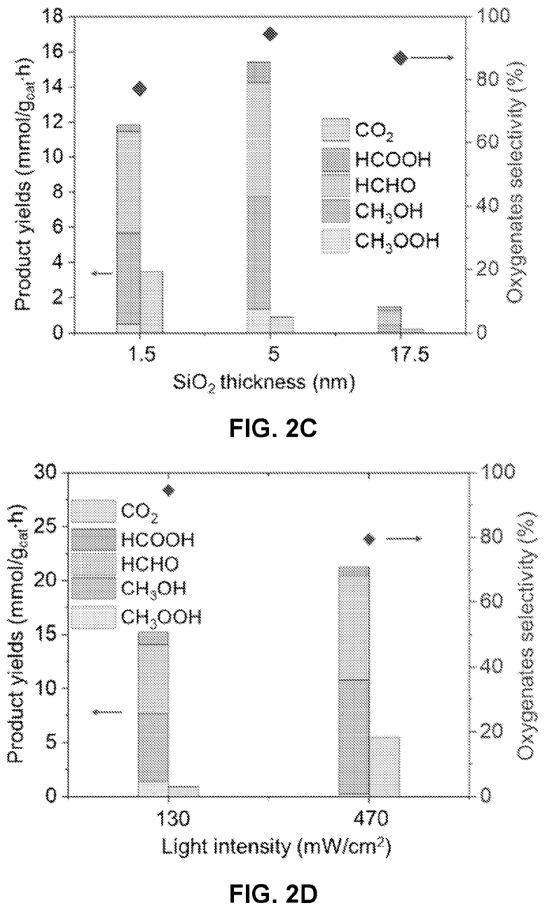

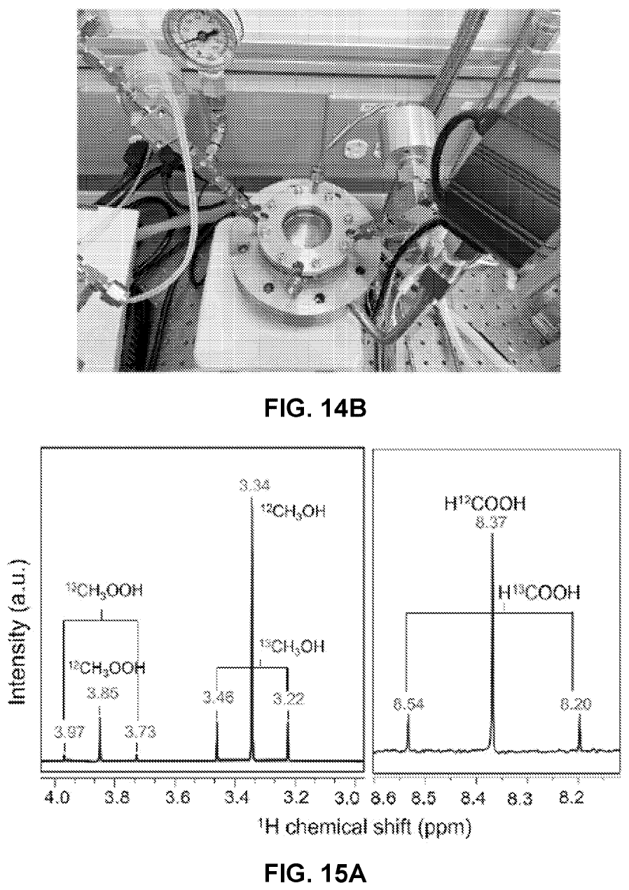

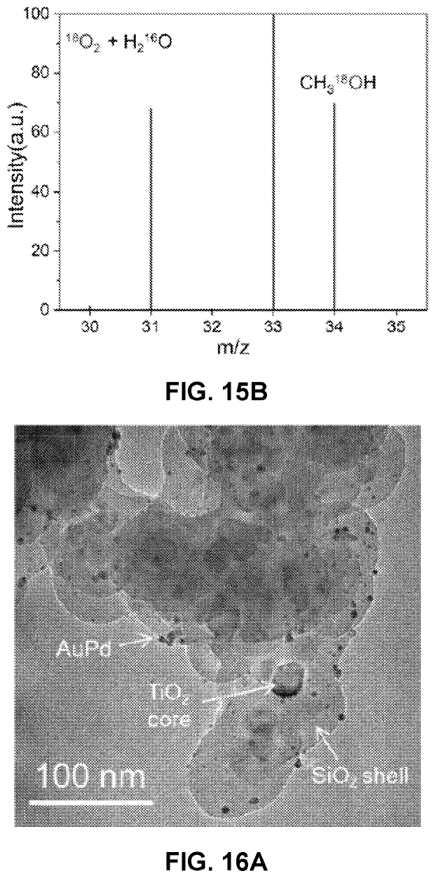

Photocatalytic experiments were performed at room temperature (25±3° C.) in a batch reactor with 6.9 bar CH₄ and 2.75 bar O₂ (FIGS. 14A-14B). Bare TiO₂ produced CO₂ and HCHO as the major products (11.06 mmol/gcat·h) with only a small amount of methanol (0.91 mmol/gcat·h). With AuPd loaded (AuPd/TiO₂), the methanol yield increased by 4.6 times (4.2 mmol/gcat·h) with 52.7% selectivity toward oxygenates (FIG. 2A). Remarkably, for TiO₂@SiO₂—AuPd, the selectivity for oxygenates increased to 94.5% while the highest total oxygenates yield was 15.4 mmol/gcat·h with methanol production at 7.7 mmol/gcat·h and CO₂ production at only 0.89 mmol/gcat·h. The formation of HCHO and HCOOH is likely due to the methanol oxidation by ·OH radicals in the bulk water solution. The apparent quantum yield (AQY) was determined to be 2.45% at 365 nm (Table 3). Experiments using isotope labeled ¹³CH₄ and ¹⁸O₂, along with control experiments without CH₄ or light, confirmed that the carbon and oxygen in the oxygenates originated from CH₄ and O₂, respectively (FIGS. 15A-15B, Table 2). These findings are consistent with previous studies in other photocatalytic systems.

TABLE 2
Product yields of various catalysts for direct oxidation
of CH₄ using O₂ as the oxidant in water

| | | | | | | | | All |
|---|---|---|---|---|---|---|---|---|
| | | Amount of product (μmol) | | | | | | products |
| Entry | Catalyst | CH₃OOH | CH₃OH | HCHO | HCOOH | C₂H₆ | CO₂ | (μmol) |
| 1 | TiO₂@SiO₂—AuPd | 13.7 | 63.8 | 65.2 | 11.7 | 0 | 8.9 | 163.3 |
| 2 | TiO₂ | 0 | 9.1 | 58.8 | 0 | 0 | 51.8 | 119.7 |
| 3 | AuPd/TiO₂ | 9.5 | 32.5 | 34.8 | 0.8 | 0 | 69.6 | 147.2 |
| 4 | TiO₂@SiO₂ | 2.3 | 1.4 | 5.3 | 0 | 0 | 0.6 | 9.6 |
| 5 | SiO₂ | 0.4 | 0.1 | 1.3 | 0 | 0 | 0 | 1.8 |
| 6 | AuPd/SiO₂ | 1.1 | 1.5 | 1.9 | 0 | 0 | 0.5 | 5.0 |
| 7 | AuPd/SiO₂ and | 5.4 | 4.9 | 8.2 | 0.8 | 0 | 0.9 | 20.2 |
| | AuPd/TiO₂ | | | | | | | |
| | physical mixture | | | | | | | |
| 8^{a} | TiO₂@SiO₂—AuPd | 0 | 0 | 0 | 0 | 0 | 0 | 0 |
| 9^{b} | TiO₂@SiO₂—AuPd | 0 | 0 | 0 | 0 | 0 | 0 | 0 |
| 10^{c} | — | 0 | 0 | 0 | 0 | 0 | 0 | 0 |

Reaction conditions (unless otherwise specified): 10 mg photocatalyst, 100 mL H₂O, 6.9 bar CH₄, 2.75 bar O₂, 26 ± 3° C. reaction temperature, 1 h reaction time, light source: 365 nm UV LED with light intensity of 130 mW/cm².
^{a}Entry 8: reaction is performed without light illumination (dark condition).
^{b}Entry 9: 6.9 bar Ar gas instead of CH₄ (no CH₄ in the system).
^{c}Entry 10: no photocatalyst used.

TABLE 3
Apparent quantum yields (AQY) for various photocatalysts

| | | Amount of product (μmol) | | | | | |
|---|---|---|---|---|---|---|---|
| Catalyst | N(photons)^{a} | CH₃OOH | CH₃OH | HCHO | HCOOH | CO₂ | AQY(%)^{b} |
| TiO₂@SiO₂—AuPd | 1.7 × 10²² | 13.7 | 63.8 | 65.2 | 11.7 | 8.9 | 2.45 |
| TiO₂ | 1.7 × 10²² | 0 | 9.1 | 58.8 | 0 | 51.8 | 2.79 |
| AuPd/TiO₂ | 1.7 × 10²² | 9.5 | 32.5 | 34.8 | 0.8 | 69.6 | 3.23 |
| TiO₂@SiO₂ | 1.7 × 10²² | 2.3 | 1.4 | 5.3 | 0 | 0.6 | 0.13 |

Reaction conditions: 10 mg TiO₂@SiO₂—AuPd, 100 mL H₂O, 6.9 bar CH₄, 2.75 bar O₂, 25 ± 3° C. reaction temperature, 1 h reaction time, light source: 36 5 nm UV LED.
^{a}Light intensity: 0.13 W/cm², irradiation area: 19.625 cm², light incident time: 1 h
^{b}AQY(%) = [n(CH₃OOH) + n(CH₃OH) × 3 + n(HCHO) × 5 + n(HCOOH) × 7 + n(CO₂) × 9] × 100%/N(photons), where n(CH₃OOH), n(CH₃OH), n(HCHO), n(HCOOH) and n(CO₂) represent the mole numbers of produced CH₃OOH, CH₃ OH, HCHO, HCOOH and CO₂ molecules, respectively. For example, the AQY for TiO₂@SiO₂—AuPd is calculated as: (13.7 + 63.8 × 3 + 65.2 ×+ 11.7 × 7 + 8.9 × 9) × 10⁻⁶ × 6.02 × 10²³ × 100%/1.7 × 10²² = 2.45%.

The critical role of amorphous SiO₂ shell in mitigating methanol overoxidation in photochemical methane conversion was further demonstrated by decreasing the water volume (FIG. 2B3), as the presence of water is known to stabilize methanol and prevent its overoxidation to CO₂. After decreasing water volume from 100 to 20 mL, the oxygenates selectivity over AuPd/TiO₂ decreased from 52.7% to 18.3% with CO₂ as the dominant product. Remarkably, TiO₂@SiO₂—AuPd largely maintained the high selectivity toward oxygenates (82.6%). Here, a 100 mL water volume was used for all following studies.

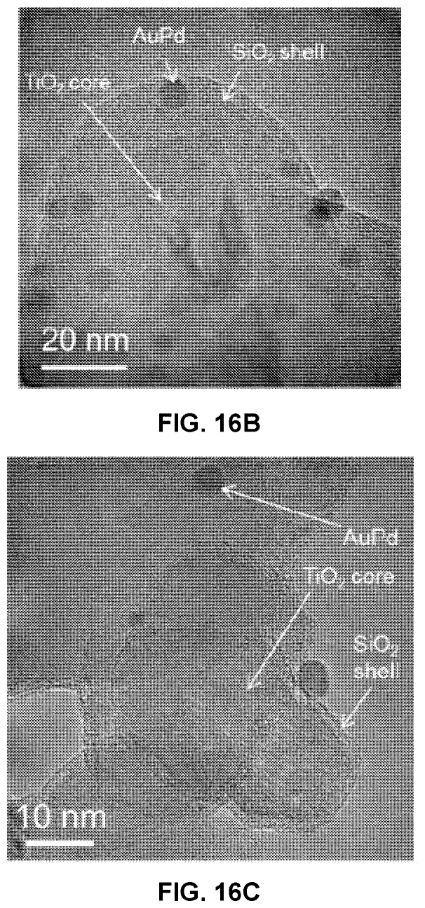

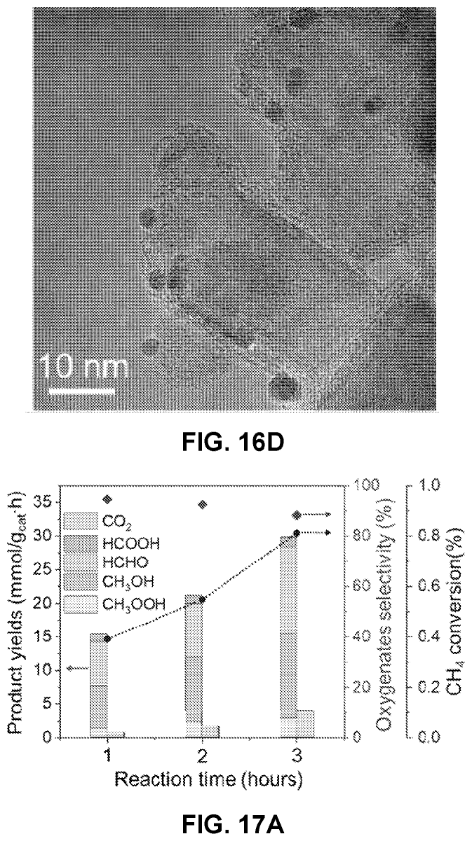

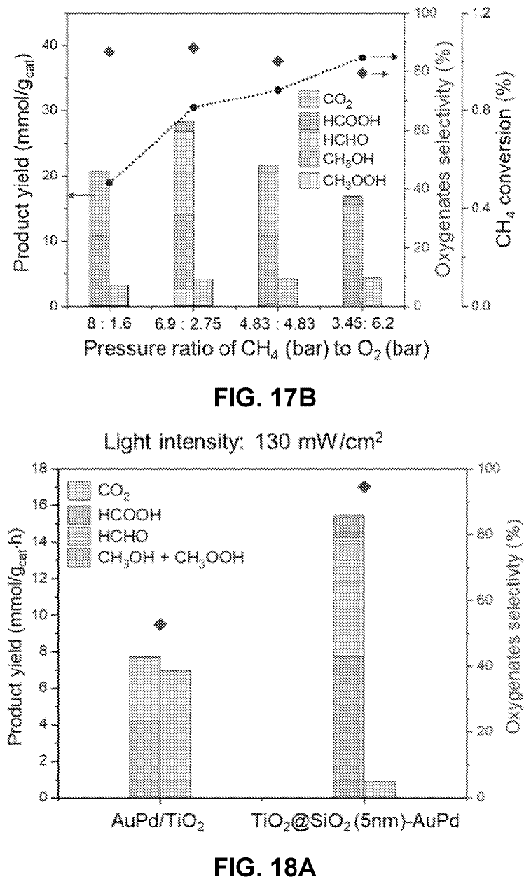

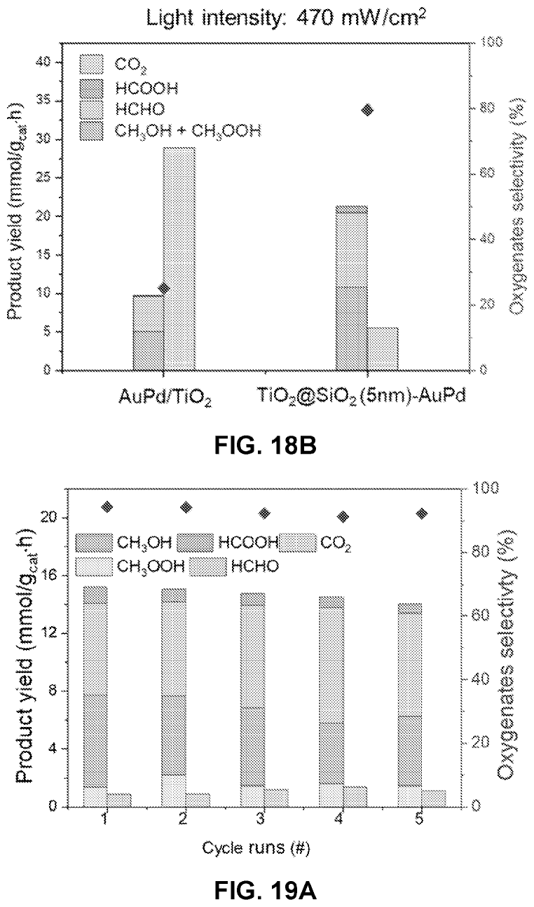

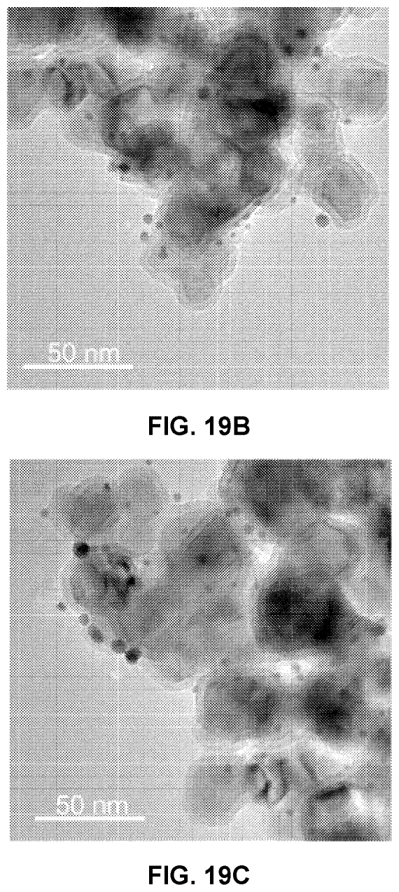

The silica shell thickness is an essential parameter for the catalyst design. With 6.9 bar of methane and 2.75 bar of oxygen, a 5 nm thick silica shell produces the optimal oxygenates selectivity and yields (FIG. 2C). A thicker shell of 17.5 nm produces a lower oxygenate yield, while a thinner shell of 1.5 nm thickness cannot effectively suppress methanol overoxidation (FIGS. 2C and 16A-16D). The reactions conditions were also varied to investigate their impact on the oxygenates yields and selectivity. The oxygenates yield increased with irradiation time (FIG. 17A): methane conversion reached 0.8% after 3 h of reaction time while maintaining 90% oxygenates selectivity. When the CH₄ partial pressure decreased from 6.9 to 3.45 bar and the O₂ partial pressure increased from 2.75 to 6.2 bar O₂, methane conversion further increased to 1% after 3 h of irradiation (FIG. 17B). In addition, further increasing the light flux to 470 mW/cm², 21.3 mmol/gcat·h of oxygenates was produced with 80% oxygenates selectivity (FIG. 2D). Comparably, without the silica coating, the selectivity of AuPd/TiO₂ catalyst dropped to 25% at this light flux (FIGS. 18A-18B). Finally, TiO₂@SiO₂—AuPd exhibited high stability over five operation cycles with no obvious structural changes observed (FIGS. 19A-19C).

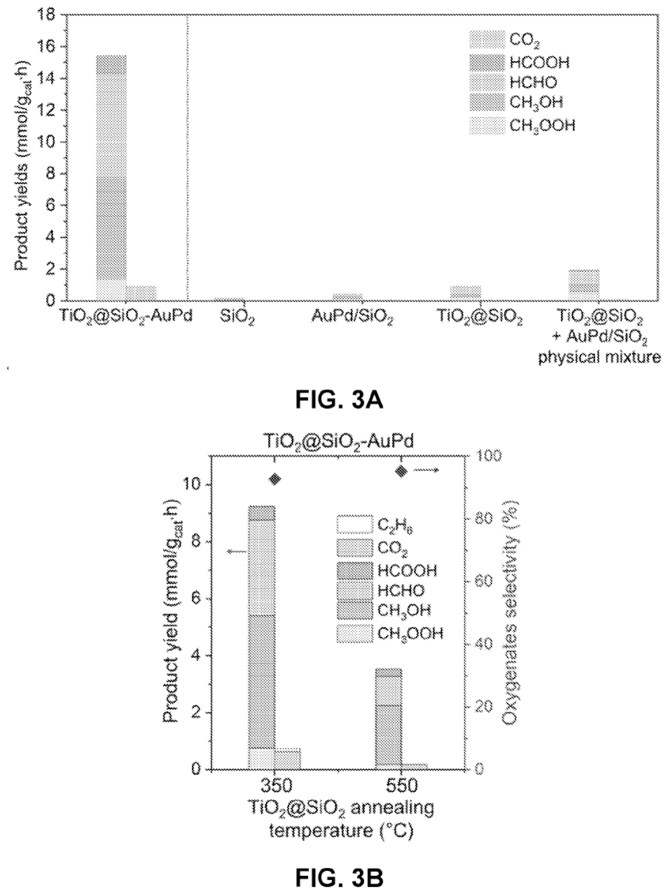

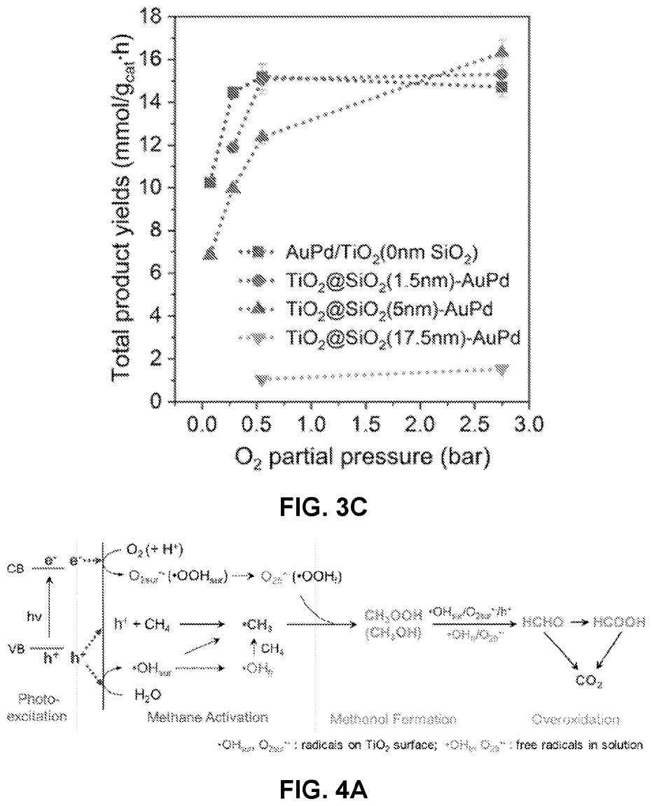

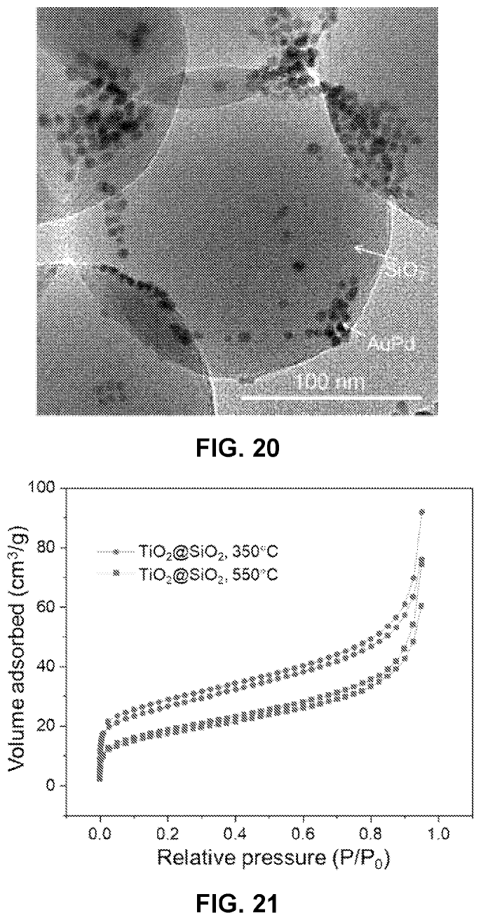

To uncover the role of silica shell and the importance of precise structural design of TiO₂@SiO₂—AuPd, the role of each component was investigated individually. FIG. 3A shows catalytic performance of the single interface catalysts (SiO₂, AuPd/SiO₂, and TiO₂@SiO₂). SiO₂ and AuPd/SiO₂ exhibit little photocatalytic activity, suggesting that SiO₂ is catalytically inert (FIGS. 3A and 20). TiO₂@SiO₂ alone also gives low oxygenates yields 12 times lower compared to that of bare TiO₂. The low photocatalytic activity of TiO₂@SiO₂ confirms that electron/hole migration from TiO₂ surface through the insulating SiO₂ layer does not occur. This also indicates methane and/or O₂ permeation is largely blocked by the SiO₂ shell; otherwise TiO₂@SiO₂ would have similar production as TiO₂. Decorating AuPd on TiO₂@SiO₂ promotes methanol production compared to TiO₂ (FIG. 3A), indicating that AuPd enables the key species being blocked by silica to carry out its role. Compared to bare TiO₂ and TiO₂@SiO₂—AuPd, the physical mixture of TiO₂@SiO₂ and AuPd/SiO₂ also possesses low catalytic activity (FIG. 3A), showing that the promoting effect of AuPd requires it to be in spatial proximity to TiO₂. This experiment is also consistent with the SiO₂ thickness dependence experiments, where increased spatial separation between AuPd and TiO₂ to 17.5 nm readily gives lowered yield (FIG. 2C). In addition, increasing the calcination temperature of TiO₂@SiO₂ to 550° C. during the synthesis of TiO₂@SiO₂—AuPd, which makes the silica layer denser and nonporous, also results in a substantial decrease in methanol production (FIGS. 3B and 21). Thus, silica and AuPd synergistically act as a functional transport mediating shell for the TiO₂ photocatalyst and give high reaction yields and selectivity.

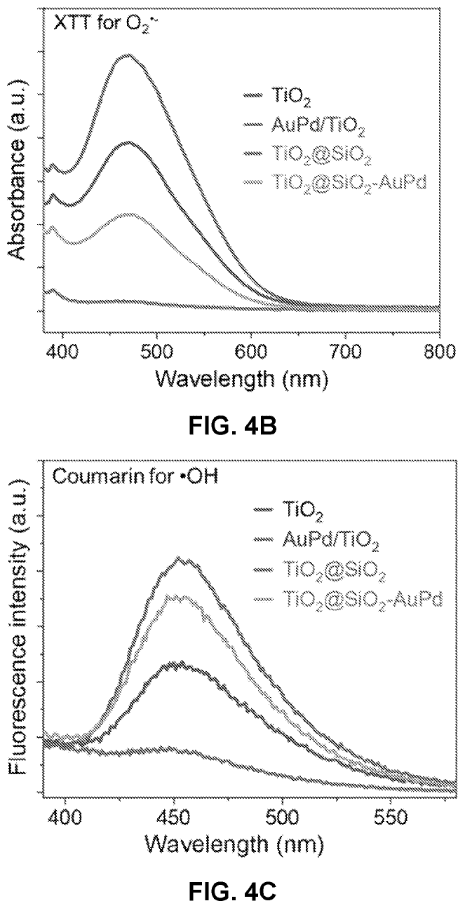

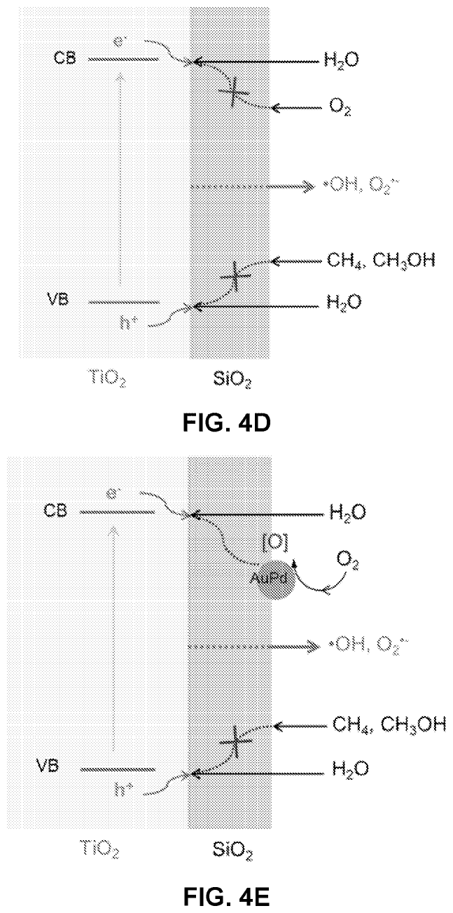

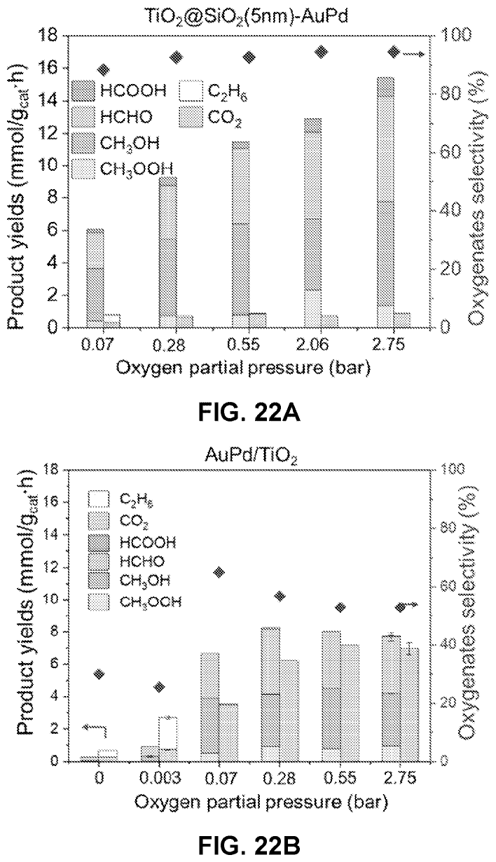

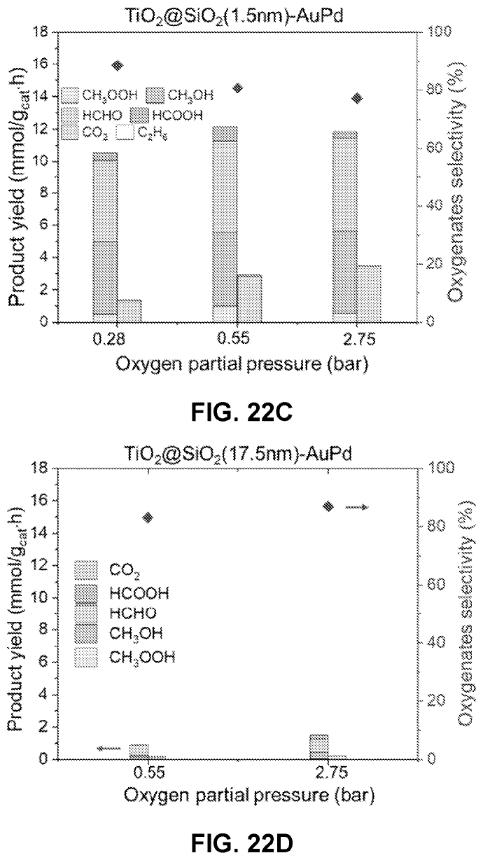

The key species involved in this photochemical process could be water that reacts with photogenerated h⁺ or oxygen that reacts with photogenerated e⁻, and ·OH/O₂·⁻ radicals that diffuse out of the silica shell (FIG. 4A). Water should be permeable through SiO₂ due to the highly hydrophilic nature of silica. Considering the fact that AuPd outside silica cannot directly affect the diffusion of radicals from the TiO₂/SiO₂ interface, the key species that is affected by AuPd can only be oxygen (FIG. 4A, methane activation stage). This speculation is confirmed by oxygen pressure dependence of catalysts with different silica shell thickness (FIGS. 3C and 22A-22D). Increasing O₂ partial pressure gives higher total yields for TiO₂@SiO₂—AuPd, indicating more ·OH/O₂·⁻ radicals were produced to activate methane. For thicker silica shell, higher oxygen pressure is required to reach the optimal yield, indicating the key species that AuPd allows to diffuse through silica shell is oxygen. Enhanced oxygen diffusion through SiO₂ would promote electron scavenging, consistent with the increased reaction yield (FIG. 4A).

Considering that such oxygen diffusion, not observed on the TiO₂@SiO₂, was enabled by loading AuPd nanoparticles on the silica shell, it is hypothesized that AuPd enables oxygen to permeate the silica shell by dissociating the O₂ molecule. AuPd nanoparticles supported on metal oxides have previously been reported to facilitate O₂ molecule dissociation. In addition, it is known that the transport of atomic oxygen species through oxide thin film at room temperature is dominated by a field-induced drift, which is generated by the chemisorption of reactive oxygen species. Although no direct evidence was obtained for such seemingly counterintuitive oxygen dissociation process, it is the most plausible rationale for the series of results presented above.

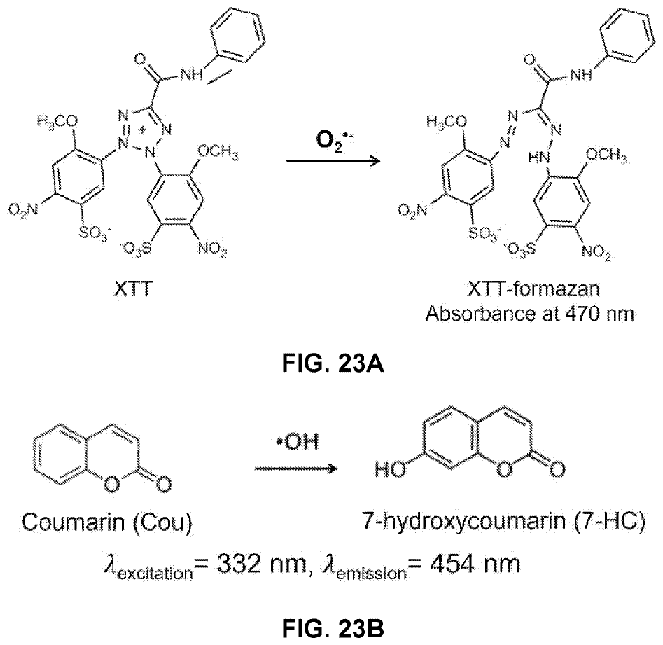

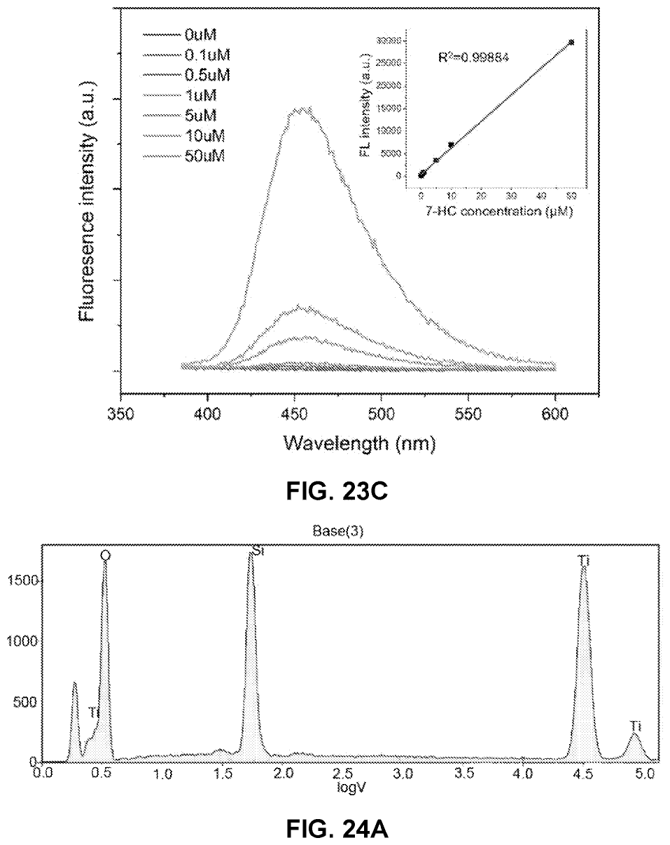

We further confirmed the effect of the SiO₂ shell and AuPd by measuring the radical generation with different catalysts (FIGS. 4B-4C). As widely reported in previous literature reports, O₂·⁻ radicals formed by photochemical oxygen reduction can be captured by 2,3-bis(2-methoxy-4-nitro-5-sulfophenyl)-2H-tetrazolium-5-carboxanilide inner salt (XTT salts) to produce a formazan product with absorption at 470 nm (FIG. 23A), allowing for qualitative comparison of photogenerated radical yield between different photocatalysts. With XTT, absorption at 470 nm was observed for TiO₂ and AuPd/TiO₂ while little absorption was observed for TiO₂@SiO₂, indicating there is little O₂·⁻ radical production on TiO₂@SiO₂. As the reduction of oxygen molecules takes place on the surface of TiO₂, this result suggests that the silica shell prohibits oxygen molecules from accessing the TiO₂ surface. This is also consistent with the observed low O₂ diffusion coefficient in SiO₂ at room temperature. However, with AuPd loaded on TiO₂@SiO₂, the XTT absorbance increased significantly (FIG. 4B), indicating the addition of AuPd promotes the transport of oxygen through the SiO₂ shell to scavenge electrons and generate O₂·⁻ radicals. This also suggests that O₂·⁻ radicals can diffuse through SiO₂ shell. The production of ·OH radicals formed by water reacting with holes was also studied using coumarin as a fluorescence probe (FIGS. 4C and 23B-23C), which exhibits a similar trend as O₂·⁻ radical production.

As evidenced from the results presented, in accordance with previous studies in photochemical reactions, the primary working principle of TiO₂@SiO₂—AuPd can be hypothesized (FIGS. 4A and 4E). Water molecules permeate the SiO₂ shell, diffuse to the TiO₂ surface, and are oxidized by photogenerated holes to form ·OHₛᵤᵣ, which subsequently diffuses out to the bulk solution. In addition, O₂ molecules, blocked by SiO₂, are dissociated by AuPd to atomic oxygen, which diffuse into the SiO₂ shell to scavenge the photo-generated electrons. Hydroxyl radicals in the solution (i.e., ·OH_{fr}) react with methane to produce ·CH₃, which then reacts with oxygen species on AuPd to produce methanol. The produced methanol is blocked from contacting the TiO₂ surface by the amorphous SiO₂ shell and therefore is not further oxidized to CO₂ by holes and ·OHₛᵤᵣ on the TiO₂ surface, which increases both the oxygenates selectivity and overall methanol production. It is worth noting that methanol can still be oxidized by the ·OH_{fr} and O2_{fr}·⁻ radicals in the bulk water solution, leading to the formation of HCHO and HCOOH. The deep oxidation to CO₂ is largely prevented due to solvation of water molecules around methanol. The results also indicate that, in the photocatalytic methane oxidation in water, methane activation is majorly induced by ·OH and O₂·⁻ in the solution instead of holes on TiO₂ surface. However, it is worth noting that water could still be a participant in the electron scavenging when atomic oxygen species are in low supply.

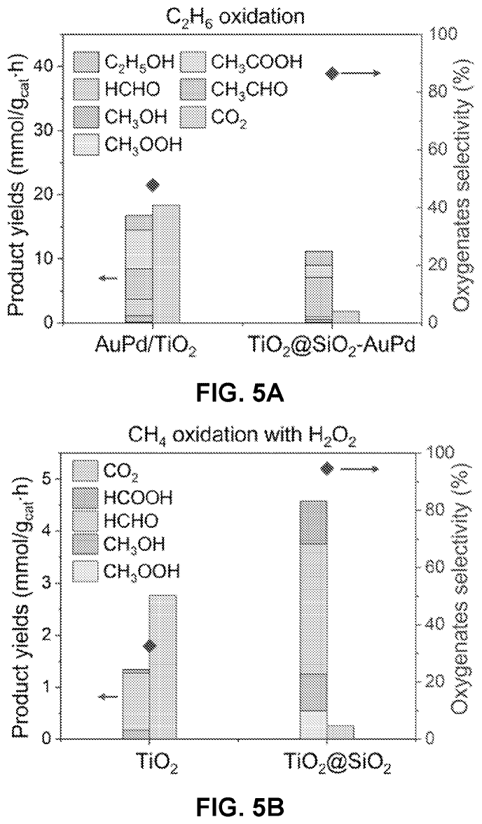

Enclosing the photocatalyst with a water permeable shell is a generalizable pathway to increase the selectivity of various photo-oxidative reactions, which could have a broad impact in the field of clean energy. The potential of the disclosed strategy was demonstrated in the selective transformation of ethane, which is another major component of natural gas. FIG. 5A shows that the TiO₂@SiO₂—AuPd suppressed CO₂ production by a factor of 10 and improved liquid oxygenates selectivity from 47% to 86% compared to AuPd/TiO₂. This concept can also be applied in designing noble-metal-free catalysts. When H₂O₂ is used as the oxidant instead of O₂, it can readily scavenge electrons to produce ·OH. FIG. 5B shows that with 6.9 bar of methane and 5 mM H₂O₂, TiO₂@SiO₂ produced liquid oxygenates at 95% selectivity with more than 7 times higher methanol yield compared to bare TiO₂, which produced CO₂ as the dominant product. Hence, the silica shell without AuPd can also promote methanol selectivity with H₂O₂ as the oxidant, suggesting the possibility of using other oxidants.

In summary, an encapsulated photocatalyst with transport selective architectures was designed as a generalizable strategy to achieve high selectivity and activity simultaneously in photochemical alkane oxidation reactions. The transport selective architecture employs a nanometer thick, water permeable oxide shell to prevent photogenerated holes from overoxidizing the oxygenates products and AuPd nanoparticles to enable the diffusion of oxygen as electron scavenger. It is believed that this strategy demonstrates the power of precise nanostructure design for photocatalysis and can be widely applied to catalytic reactions where the decoupling of surface photochemical processes and solution chemical processes is desirable.

### Example 2: Materials and Methods

### Materials

All reagents were commercially obtained without purification. Titanium (IV) oxide (P25), Tetraethyl orthosilicate (TEOS), gold chloride trihydrate (HAuCl₃·3H₂O), palladium chloride (PdCl₂), sodium borohydride (NaBH₄), polyvinyl pyrrolidone (PVP, Mw=130,000), ammonia solution (28-30%), hydrochloric acid (37%), ethanol, dimethyl sulfoxide (DMSO) were purchased from Sigma-Aldrich. Methane (99.999% research purity), oxygen (99.999%) and argon (99.999%) were purchased from Airgas. Deionized (DI) water with a resistivity of 18.2 MΩcm⁻¹ was used in all experiments.

### Synthesis of TiO₂@SiO₂

TiO₂@SiO₂ particles are synthesized using modified procedures from earlier work. In a typical synthesis run, 50 mg of commercial TiO₂ (P25) powder was dispersed in 25 mL of a mixed solvent of water and ethanol (volume ratio of H₂O:EtOH=1:4). A certain amount of TEOS was added to suspension. After sonication for 15 mins, 0.5 mL ammonium hydroxide solution (28-30%) was added into the mixture to catalyze the hydrolysis of TEOS. The reactor was stirred at room temperature overnight. The TiO₂@SiO₂ samples were collected via centrifuge and washed twice using DI water. TiO₂@SiO₂ sample were then calcinated under 350° C. for 2 hours in air, unless specified. For the TiO₂@SiO₂ (550° C.) sample, the synthesis procedure was the same but had a different calcination condition: 550° C. 4 h in air. The thickness of SiO₂ was well-controlled by tuning the added TEOS amount; 100 μL TEOS was added to obtain SiO₂ shell with ˜5 nm thickness.

### Synthesis of AuPd Nanoparticles

The AuPd nanoparticles were synthesized using procedures from previous work. Typically, 12.6 mg HAuCl₃·3H₂O and 5.67 mg PdCl₂ (Au and Pd molar ratio at 1:1) and 11.6 mg PVP (Mw=130,000) were dissolved in 400 mL water. After stirring for 30 minutes, 3.2 mL freshly made NaBH₄ (0.1 M) aqueous solution was injected into the above solution. After stirring for another 2 hours, the AuPd colloid was concentrated to 100 mL and stored for future use. The concentration of AuPd NPs solution was measured by inductively coupled plasma-optical emission spectroscopy (ICP-OES).

### Synthesis of TiO₂@SiO₂—AuPd

100 mg as-synthesized TiO₂@SiO₂ powder was dispersed in 30 mL water under sonication. A controlled amount of the AuPd colloid solution was added dropwise to the above solution (the volume of AuPd nanoparticle solution was calculated by the loading and the concentration of AuPd nanoparticle solution). The mixture was stirred at room temperature overnight. The TiO₂@SiO₂—AuPd samples were collected by centrifuge and then annealed in air at 350° C. for 2 h. The SiO₂ shell thickness was 5 nm for TiO₂@SiO₂—AuPd, unless otherwise specified.

### Synthesis of AuPd/TiO₂ and AuPd/SiO₂

The synthesis procedure was same as the synthesis of TiO₂@SiO₂—AuPd, except using TiO₂ or SiO₂ as supporting materials.

### Materials Characterization

Transmission electron microscopy (TEM) was performed on a FEI Tecnai transmission electron microscope with an acceleration voltage of 200 kV. High resolution TEM, scanning transmission electron microscopy (STEM) and energy dispersive spectrometer were performed on a FEI Titan electron microscope with an accelerating voltage of 300 kV. X-ray diffraction (XRD) data were collected on an Empyrean X-ray diffractometer from PANalytical B.V. with Cu Kα (λ=1.5418 Å). The molar ratio of Au, Pd and mass loading of AuPd co-catalysts were determined by inductively coupled plasma-optical emission spectrometry (ICP-OES) analysis (Thermo Scientific ICAP 6300 Duo View Spectrometer). Nitrogen sorption isotherms were measured using an Anton PaarAutosorb iQ3 system. UV-vis diffuse reflectance spectra (UV-DRS) were measured by an Agilent Cary 6000i UV/Vis/NIR spectrometer and transformed into absorption spectra via Kubelka-Munk transformation. X-ray absorption near edge structure (XANES) and extended X-ray absorption fine structure (EXAFS) experiments at Au L3-edges and Pd K-edge were carried out in the fluorescence mode at the beamline 20-ID of Advanced Photon Source at Argonne National Laboratory. The incident beam was monochromatized by using a Si (111) fixed-exit, double-crystal monochromator, a harmonic rejection mirror was applied to cut off the harmonics at high X-ray energy. Data reduction, data analysis, and EXAFS fitting (Table 4) were performed with the Athena and Artemis software packages.

TABLE 4
EXAFS fitting parameters of the TiO₂@SiO₂—AuPd photocatalyst

| Bond | CN | R/R_{Reff} (Å) | σ² (Å²) |
|---|---|---|---|
| Au—O | 0.39 | 1.94/1.90 | 0.00690 |
| Au—Pd | 2.27 | 2.82/2.81 | 0.01233 |
| Au—Au | 8.73 | 2.82/2.81 | 0.01167 |

EXAFS fitting range: k range: 3-12.6 Å⁻¹; R range: 1.3-3.5 Å; k²-weighted; ΔE (eV) is 4.9; R factor is 0.018.
The amplitude reduction factor S₀² was determined to be 0.8 through fitting the FT-EXAFS of standard Au foil which is measured simultaneously during the experiment. CN, the average coordination number; R, as-fitted distance between absorber and backscatter atoms; RReff, the distance between absorber and backscatter atoms in model; σ², Debye-Waller factor to describe the variance due to structural disorder (both lattice and thermal); ΔE, threshold Energy Correction; R factor (%) indicates the goodness of the fit.

### Catalyst Tests

The photocatalytic reactions were performed in a 250 mL batch photoreactor equipped with a quartz window to allow for light irradiation (FIGS. 14A-14B). In a typical run, 10 mg catalyst was dispersed in 100 mL water by ultrasonication for 30 minutes. The sample solution was then placed in a Teflon lined vessel in the photoreactor and degassed for 30 min to remove air. The reactor vessel was then pressurized with the desired amount of methane (99.999%, Airgas) and oxygen (99.999%, Airgas) with total pressure of 9.65 bar (140 psi). The maximum pressure rating for the reactor was 10 bar (i.e., 145 psi), thus the reactions were always carried out under 9.65 bar for safety reasons. The temperature was monitored via a thermometer inserted in the reaction solution and was maintained to be 25° C.±3° C. during the reaction. The reaction occurred under vigorous stirring at 700 rpm. A 365 nm UV LED array was used as the light source and the light intensity was measured to be 130 mW/cm². After the reaction (usually 1 h), the reactor was cooled in an ice bath to <10° C. for product analysis. The light intensity of the irradiation light was measured by a Thorlab integrating sphere photodiode power sensor.

### Product Analysis

The photoreactor was directly connected to a gas chromatograph (SRI instrument MG #5), equipped with a thermal conductivity detector (TCD), a flame ionization detector (FID) and a methanizer for the gas product analysis of CO₂, CO, and ethane. The liquid oxygenate products were analyzed using nuclear magnetic resonance spectroscopy (NMR) and the colorimetric method. CH₃OH, CH₃OOH and HCOOH were quantified via ¹H-NMR on a Varian Inova 600 MHz NMR equipped with a water suppression system. Typically, 0.63 mL of liquor was mixed with 0.17 mL of D₂O to prepare a solution for NMR measurements. Dimethyl sulfoxide (DMSO) was used as an internal standard. The formaldehyde (HCHO) amount was determined by the colorimetric method. First, 15 g ammonium acetate, 0.3 mL of acetic acid, and 0.2 mL pentane-2,4-dione were dissolved in 100 mL water, to make the reagent solution. Then, 0.5 mL of the sample liquor was mixed with 2 mL of water and 0.5 mL of reagent solution. The mixture was kept at 35° C. for 1 hour in a water bath and measured by UV-vis absorption spectroscopy at 412 nm (Agilent Cary 6000i UV/Vis/NIR spectrometer). The concentration of HCHO in the sample liquor was determined by the calibration curve using a series of standard HCHO solutions.

The methane conversion and oxygenates selectivity in this process are calculated according to the following equations:

$$Conversion ( C {H}_{4} ) = \frac{{mol}_{total formed products}}{{mol}_{i n itial methane}} \times 10 \; 0 \%$$

$$Oxygenate product selectivity = \frac{{mol}_{produced {CH}_{3} OH , {CH}_{3} OOH , HCHO , and HCOOH}}{{mol}_{total formed products}} \times 100 \%$$

The initial methane amount in the system is calculated according to the following equation (V is the volume of the headspace of the reactor):

$$Initial methane ( mol ) = \frac{{P}_{m e t h a n e} \times V}{R \times T ( 298 K )}$$

The apparent quantum yield (AQY) was calculated according to the following equation:

$$AQY = \frac{N ( electrons )}{N ( photons )} \times 100 \%$$

where N(electrons) and N(photons) represent the number of reacted electrons and the number of incident photons, respectively. N(photons)=IAt/Eλ, where I, A, t and Eλ represent incident light intensity (W/cm²), irradiation area (cm²), light incident time (s) and photo energy (J), respectively. For the calculation of N(electrons), an approach following reported work was used: N(electrons)=n(CH₃OOH)+n(CH₃OH)×3+n(HCHO)×5+n(HCOOH)×7+n(CO₂)×9, where n(CH₃OOH), n(CH₃OH), n(HCHO), n(HCOOH) and n(CO₂) represent the mole numbers of produced CH₃OOH, CH₃OH, HCHO, HCOOH and CO₂ molecules, respectively.

### Catalyst Cycling Test

To study the reusability of the catalyst, the solid catalyst was separated by centrifugation after each reaction run. The catalysts were re-used in the next run after drying at 90° C. overnight under vacuum and annealed at 300° C. in air to remove any adsorbed organic species.

### Isotope Labeling Experiments

In the ¹³CH₄ isotopic experiments, 10 mg TiO₂@SiO₂—AuPd photocatalyst were dispersed in 20 mL H₂O and degassed for 30 min to completely remove air. 1.73 bar (25 psi) ¹³CH₄ (99 atom % ¹³C, Sigma Aldrich), 5.17 bar (75 psi) ¹²CH₄ and 2.75 bar O₂ were added to the photoreactor. The reaction was carried for 1 h under light irradiation (130 mW/cm²). The liquid products were collected and measured by ¹H-NMR (Varian Inova 600 MHz).

In the ¹⁸O₂ isotopic experiments, 10 mg TiO₂@SiO₂—AuPd photocatalyst was dispersed in 100 mL H₂O and degassed for 30 min to completely remove air. 8.27 bar (120 psi) CH₄ and 1.38 bar (20 psi) ¹⁸O₂ (99 atom % ¹⁸O, Sigma Aldrich) were added to the photoreactor. The reaction was carried for 4 h under light irradiation (130 mW/cm₂). The products were measured by GC-MS (Agilent 7890B GC with Agilent 5977A MS).

Analysis of Photogenerated Superoxide Anion Radicals (O₂·⁻)

2,3-Bis(2-methoxy-4-nitro-5-sulfophenyl)-2H-tetrazolium-5-carboxanilide inner salt (XTT salt) was used as the indicator for the O₂·⁻ radical measurements. XTT is reduced by O₂·⁻ to form the orange-colored XTT-formazan, which was measured using a UV-vis spectrophotometer at 470 nm. Typically, 10 mg of catalyst was dispersed in 100 mL of a 0.1 mM XTT aqueous solution in dark under stirring. Argon was purged through the reactor several times to remove air. 6.90 bar (100 psi) argon (99.999%, Airgas) and 0.34 bar (5 psi) O₂ (21% O₂ balanced with Ar, Airgas) were added into the reactor. After irradiation for 10 mins, the reactant solution was collected by centrifugation and then used for UV-Vis measurements.

### Analysis of Photogenerated Hydroxyl Radicals (·OH)

The hydroxyl radicals (·OH) production was measured by photoluminescence (PL) experiments using coumarin as a probe molecule. Coumarin reacts with ·OH to form 7-hydroxycoumarin (7-HC), which gives high fluorescence at around 454 nm (FIG. 20). The fluorescence intensity of 7-HC is proportional to the amount of ·OH generated. Typically, 10 mg of catalyst was dispersed in 100 mL of a 1.0 mM coumarin aqueous solution in dark under stirring. Argon was purged through the reactor several times to remove air. 6.90 bar (100 psi) argon (99.999%, Airgas) and 0.34 bar (5 psi) O₂ (21% O₂ balanced with Ar, Airgas) were added into the reactor. After irradiation for 10 mins, the reaction solution was collected by centrifugation and then used for fluorescence measurements. The spectra were measured by fluorescence spectrophotometer (Horiba FluoroLog-3 spectrofluorometer) with excitation wavelength at 332 nm.

### Example 3: Turnover and Reaction Mechanism

### Turnover Number (TON) Calculation

TON is calculated via the following formula:

$$TON = \frac{moles of total products}{moles of active sites}$$

As there are no well-established techniques for precisely measuring the number of active sites in heterogeneous photocatalysts, the number of moles of catalysts is used to represent the moles of active sites according to the reported work. It is worth noting that the calculated TON using this method is a lower limit for TON since the number of active sites in moles is lower than the amount of catalyst in moles.

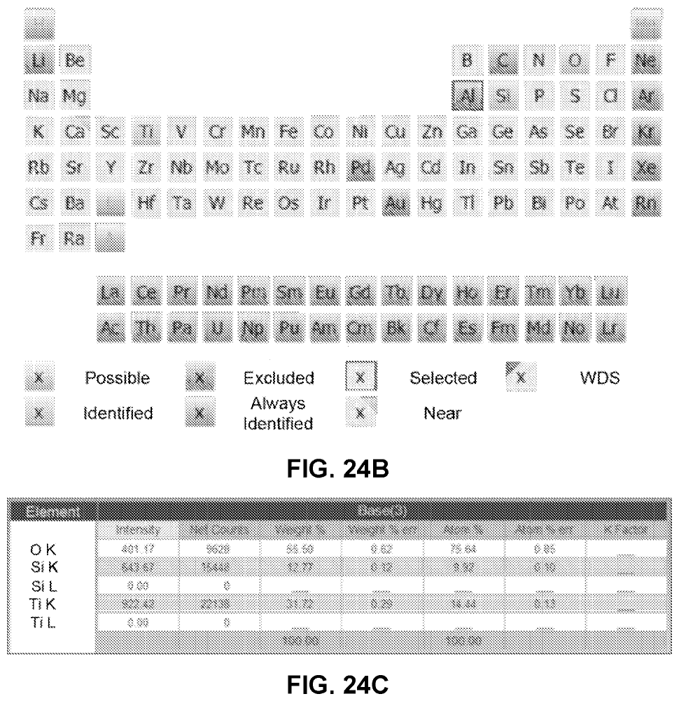

The mole numbers of total products after 3 h reaction for 10 mg TiO₂@SiO₂—AuPd catalysts is 339.2 μmol (FIG. 17A). The mass loading for AuPd in this example is 1 wt. % (Table 1). According to SEM-EDS data (FIG. 24), the weight ratio of Ti/Si in the catalysts is 2.49, so that the weight percentages of TiO₂, SiO₂, and AuPd in the catalysts are 65 wt %, 34 wt %, and 1 wt. % respectively. The number of moles of TiO₂, SiO₂, and AuPd in the catalysts are calculated to be 81.2 μmol, 56.7 μmol, and 0.7 μmol, respectively.

It has been demonstrated that SiO₂ is catalytically inert for the reaction (FIG. 3A), thus the moles of TiO₂ were used as the number of moles of active sites. The TON is calculated to be 4.2. If the total number of moles of catalyst (81.2 μmol+56.7 μmol+0.7 μmol=138.6 μmol) are used, the TON is calculated to be 2.4, which is also larger than unity. These calculations clearly show that the process is a catalytic process.

### Reaction Mechanism Discussion

We first find that the total yields as well as the O₂·⁻ radicals (or ·OOH radicals) production are strongly suppressed by the SiO₂ shell, which is alleviated by the addition of AuPd (FIG. 4B). Therefore, it is hypothesized that the electrically insulating silica shell prohibits both photoexcited electrons from transporting through its thickness and oxygen molecules (O₂) from accessing photoelectrons at the TiO₂ surface. This prevents the reaction between photogenerated electrons and oxygen molecules. The presence of AuPd on SiO₂ is unlikely to change electron transport via the SiO₂. However, with AuPd loaded on TiO₂@SiO₂, the O₂·⁻ indicating product increased significantly (FIG. 4B), indicating the addition of AuPd promotes the production of O₂·⁻ radicals.

Given that AuPd nanoparticles supported on metal oxides has been reported to facilitate O2 dissociation, the primary role of AuPd on SiO₂ is to activate oxygen molecules to atomic oxygen species (O) so that it can penetrate the silica shell and scavenge electron from TiO₂. This is herein called “O transport mechanism,” which is discussed herein and shown in FIGS. 4A and 4D-4E.

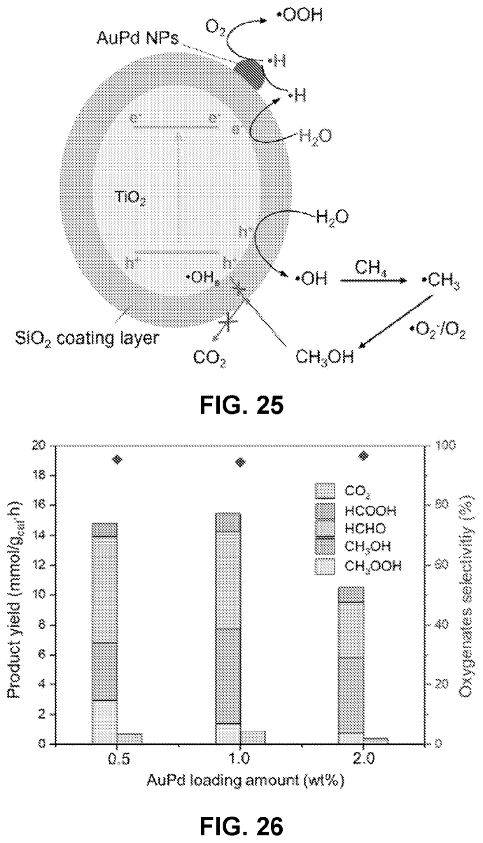

However, an alternative process was also explored. Water, instead of O₂, could serve as an electron scavenger, reacting with photogenerated electrons to form ·H atoms (reaction A2 below), which can diffuse through SiO₂ shell via spillover. These ·H atoms then react with O₂ to generate ·OOH radicals, which could be promoted by AuPd nanoparticles. Herein, this is called the “H transport mechanism” as shown in FIG. 25. Based on experimental observations, some arguments are developed to help reach some qualitative understanding of the key mechanisms. As concluded later, the experiments described herein support the mechanism of O transport.

First, considering the H-transport mechanism, the key reactions with electrons and holes on TiO₂ surface:

H₂O+h⁺→H⁺+·OH, water oxidation (A1)

H₂O+e⁻→·H+OH· (or H⁺+e⁻→H), water reduction (A2)

Consider now that H atoms and ·OH radicals diffuse from the TiO₂ surface through SiO₂ layer to be released to the bulk water solution or find AuPd catalyst surface.

·H+O₂→·OOH (facilitated by AuPd) (A3)

·H+·OH→H₂O (side reaction that eliminates ·H and ·OH) (A4)

·H+·H→H₂ (hydrogen gas production) (A5)

H₂+½O₂→H₂O (in the presence of O₂ and AuPd catalyst, H₂ and O₂ will form H₂O) (A6)

·OH+CH₄→·CH₃+H₂O (A7)

As methane is also blocked by the SiO₂ shell, it cannot be activated directly by holes on TiO₂ surface. The major contributor for methane activation is ·OH and ·OOH radicals, whose quantities are directly correlated with the total product yields as well as the final product distribution. FIGS. 22B-22C show the product yield as function of O₂ partial pressure for TiO₂@SiO₂—AuPd with 0 nm and 1.5 nm thickness, respectively. For both samples, it seems clear that the total yields and product distribution of each remains almost identical at 0.55 bar O₂ and 2.75 bar O₂. Given that reaction A3 (·H+O₂→·OOH) would be promoted by increasing O₂ partial pressure, this result can only be explained if the rate limiting factor in reaction A3 is the concentration of ·H, which could be due to the formation or the transport of ·H. When the SiO₂ thickness is increased to 5 nm, it is plausible that transport of ·H could become more of a rate limiting step than that for 1.5 nm-thick SiO₂. Hence, it should also show lack of O₂ pressure dependence, like the sample with 1.5 nm thick SiO₂. The results shown in FIG. 22A, however, does show an O₂ pressure dependence, which contradicts the H-transport mechanism. This suggests that the H-transport mechanism is likely not the dominant mechanism and that another O transport mechanism is likely responsible for the observed results.

Now let us consider the O-transport mechanism. From numerous previous works, for TiO₂—AuPd, the electron scavenging process is through oxygen reacting with photogenerated electrons. In this proposed mechanism for TiO₂@SiO₂—AuPd, the electron scavenging is carried out with oxygen. The potential reactions are as follows:

O₂→2O (in the presence of AuPd; O transports through SiO₂ to reach TiO₂ surface) (B1)

H₂O+h⁺→H⁺+·OH, water oxidation on TiO₂ surface (B2)

2O+e⁻→O²⁻, on TiO₂ surface (B3)

For AuPd/TiO₂, the product yields and distributions are almost identical for 0.55 and 2.75 bar of O₂, suggesting that concentration of O₂ is not the limiting factor of the total yields (FIG. 22B). The same holds true for the TiO₂@SiO₂—AuPd with 1.5 nm SiO₂, although product distributions are different (FIG. 22C). The lack of O₂-pressure dependence between 0.55 and 2.75 bar in these cases suggests that the electron scavenging reaction by oxygen (reaction B3) cannot be the rate limiting step for 0 nm and 1.5 nm SiO₂ cases. Hence, the only conclusion one can reach is that under these SiO₂ thicknesses and O₂ partial pressures, the likely rate limiting step is the hole water oxidation reaction (B2).

As the SiO₂ thickness is increased to 5 nm, the O₂ partial pressure dependence of the total yield emerges (FIG. 22A). This suggests that 0-transport through SiO₂ becomes competitive with hole-water reaction (reaction B2) as the rate limiting steps. This is also confirmed by increasing the photon flux in the experiments described herein. At a certain O₂ partial pressure, for TiO₂@SiO₂—AuPd catalyst, the oxygen supply to the TiO₂ surface for electron scavenging is fixed and can only keep up with the photocarrier generation to a certain rate. As a result, increasing light intensity (photocarrier generation) would have a stronger dependence for photon flux for AuPd/TiO₂ (i.e., 0 nm SiO₂) with no O-transport barrier than that for TiO₂@SiO₂—AuPd with a 5 nm thick SiO₂ layer. This is indeed the case. As shown in FIGS. 18A-18B, the total yield of AuPd/TiO₂ increases from 14.7 mmol/gcat·h to 38.7 mmol/gcat·h (by 2.6 times) after increasing the light intensity from 130 mW/cm² to 470 mW/cm², while for TiO₂@SiO₂—AuPd (5 nm thick), the total yields only increased 1.6 times (from 16.3 mmol/gcat·h to 26.8 mmol/gcat·h).

The combination of experimental results for different catalyst structures and reaction conditions suggests that in many cases the hole reaction with water (H₂O+h⁺→H⁺+·OH) could be the rate limiting step, especially in the absence or very small thicknesses of the SiO₂ shell around the TiO₂ core. The data also seem to suggest that H transport through the SiO₂ layer is unlikely a key mechanism for these photocatalytic reactions. Instead, it is likely O₂ dissociation on AuPd into O atoms and O transport through the SiO₂ shell as the key electron scavenging mechanism. Finally, depending on the thickness of the SiO₂ shell, O-transport could become competitive with hole-water reaction as the rate limiting step. For the experimental conditions investigated here, it is the combination of these two rate-limiting steps that control the total reaction yields, whereas the selectivity for partial oxygenation of CH₄ is controlled by the selective transport through the SiO₂ shell.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Agarwal, N. et al., Aqueous Au—Pd colloids catalyze selective CH₄ oxidation to CH₃OH with O₂ under mild conditions. Science 358, 223 (2017).
2. Arndtsen, B. A.; et al. Selective Intermolecular Carbon-Hydrogen Bond Activation by Synthetic Metal Complexes in Homogeneous Solution. Acc. Chem. Res. 1995, 28 (3), 154-162.
3. Bakos, T.; et al. Reactions and Diffusion of Water and Oxygen Molecules in Amorphous SiO₂. Phys. Rev. Lett. 2002, 88 (5), 055508.
4. Bau, J. A.; et al. Ultrathin Microporous SiO₂ Membranes Photodeposited on Hydrogen Evolving Catalysts Enabling Overall Water Splitting. ACS Catal. 2017, 7 (11), 7931-7940.
5. Beatty, M. E. S.; et al. Controlling the Relative Fluxes of Protons and Oxygen to Electrocatalytic Buried Interfaces with Tunable Silicon Oxide Overlayers. ACS Appl. Energy Mater. 2020, 3 (12), 12338-12350.
6. Cai, X.; et al. Unprecedentedly high efficiency for photocatalytic conversion of methane to methanol over Au—Pd/TiO₂-what is the role of each component in the system? J. Mater. Chem. A 2021, 9 (17), 10796-10802.
7. Coperet, C. C—H Bond Activation and Organometallic Intermediates on Isolated Metal Centers on Oxide Surfaces. Chem. Rev. 2010, 110 (2), 656-680.
8. Fan, Y. et al. Selective photocatalytic oxidation of methane by quantum-sized bismuth vanadate. Nat. Sustain. 2021, 4 (6), 509-515.
9. Feng, N.; et al. Efficient and selective photocatalytic CH₄ conversion to CH₃OH with O₂ by controlling overoxidation on TiO₂. Nat. Commun. 2021, 12 (1), 4652.
10. Gong, Y. et al., New insights into the photocatalytic activity of 3-D core-shell P25@silica nanocomposites: impact of mesoporous coating. Dalton Trans. 46, 4994-5002 (2017).
11. Han, S. et al, Surface Alloy Composition Controlled O₂ Activation on Pd—Au Bimetallic Model Catalysts. ACS Catal. 8, 3641-3649 (2018).
12. Huang, W.; et al. Low-Temperature Transformation of Methane to Methanol on Pd₁O₄ Single Sites Anchored on the Internal Surface of Microporous Silicate. Angew. Chem., Int. Ed. 2016, 55 (43), 13441-13445.
13. Iovino, G.; et al. O₂ Diffusion in Amorphous SiO₂ Nanoparticles Probed by Outgassing. J. Phys. Chem. C 2012, 116 (20), 11351-11356.
14. Janowicz, A. H.; et al. Carbon-hydrogen activation in completely saturated hydrocarbons: direct observation of M+R—H. fwdarw. M(R)(H). J. Am. Chem. Soc. 1982, 104 (1), 352-354.
15. Jiang, Y.; et al. Elevating Photooxidation of Methane to Formaldehyde via TiO₂ Crystal Phase Engineering. J. Am. Chem. Soc. 2022, 144 (35), 15977-15987.
16. Jin, Z.; et al. Hydrophobic zeolite modification for in situ peroxide formation in methane oxidation to methanol. Science 2020, 367 (6474), 193.
17. Labinger, J. A. Selective alkane oxidation: hot and cold approaches to a hot problem. J. Mol. Catal. A: Chem. 2004, 220 (1), 27-35.
18. Latimer, A. A.; et al. Direct Methane to Methanol: The Selectivity-Conversion Limit and Design Strategies. ACS Catal. 2018, 8 (8), 6894-6907.
19. Lieberman, R. L.; et al. Biological Methane Oxidation: Regulation, Biochemistry, and Active Site Structure of Particulate Methane Monooxygenase. Crit. Rev. Biochem. Mol. Biol. 2004, 39 (3), 147-164.
20. Luo, L.; et al. Binary Au—Cu Reaction Sites Decorated ZnO for Selective Methane Oxidation to C1 Oxygenates with Nearly 100% Selectivity at Room Temperature. J. Am. Chem. Soc. 2022, 144 (2), 740-750.
21. Luo, L.; et al. Synergy of Pd atoms and oxygen vacancies on In₂O₃ for methane conversion under visible light. Nat. Commun. 2022, 13 (1), 2930.
22. Mayer, J. M. Understanding Hydrogen Atom Transfer: From Bond Strengths to Marcus Theory. Acc. Chem. Res. 2011, 44 (1), 36-46.
23. McFarland, E. Unconventional Chemistry for Unconventional Natural Gas. Science 2012, 338 (6105), 340.
24. Metin, Ö. et al., Monodisperse gold-palladium alloy nanoparticles and their composition-controlled catalysis in formic acid dehydrogenation under mild conditions. Nanoscale 5, 910-912 (2013).
25. Murcia-López, S.; et al. Partial Oxidation of Methane to Methanol Using Bismuth-Based Photo-catalysts. ACS Catal. 2014, 4 (9), 3013-3019.
26. Nosaka, Y.; et al. Generation and Detection of Reactive Oxygen Species in Photocatalysis. Chem. Rev. 2017, 117 (17), 11302-11336.
27. Panayotov, D. A.; et al. Photooxidation Mechanism of Methanol on Rutile TiO₂ Nanoparticles. J. Phys. Chem. C 2012, 116 (11), 6623-6635.
28. Periana, R. A.; et al. Platinum Catalysts for the High-Yield Oxidation of Methane to a Methanol Derivative. Science 1998, 280 (5363), 560-564.
29. Ross, M. O.; et al. Particulate methane monooxygenase contains only mononuclear copper centers. Science 2019, 364 (6440), 566-570.
30. Schneider, J. et al., Understanding TiO₂ Photocatalysis: Mechanisms and Materials. Chem. Rev. 114, 9919-9986 (2014).
31. Schneider, J.; et al. Undesired Role of Sacrificial Reagents in Photocatalysis. J. Phys. Chem. Lett. 2013, 4 (20), 3479-3483.
32. Schwach, P.; et al. Direct Conversion of Methane to Value-Added Chemicals over Heterogeneous Catalysts: Challenges and Prospects. Chem. Rev. 2017, 117 (13), 8497-8520.
33. Serpone, N. et al. Suggested terms and definitions in photocatalysis and radiocatalysis. Int. J. Photoenergy 4, 943970, (2002).
34. Shilov, A. E. Activation of alkanes by transition metal complexes. Pure Appl. Chem. 1978, 50 (8), 725-733.
35. Snyder, B. E. R.; et al. Cage effects control the mechanism of methane hydroxylation in zeolites. Science 2021, 373 (6552), 327-331.
36. Song, H. et al., Direct and Selective Photocatalytic Oxidation of CH₄ to Oxygenates with O₂ on Cocatalysts/ZnO at Room Temperature in Water. J. Am. Chem. Soc. 141, 20507-20515 (2019).
37. Song, H.; et al. Selective Photo-oxidation of Methane to Methanol with Oxygen over Dual-Cocatalyst-Modified Titanium Dioxide. ACS Catal. 2020, 10 (23), 14318-14326.
38. Song, H.; et al. Solar-Energy-Mediated Methane Conversion. Joule 2019, 3 (7), 1606-1636.
39. Stilhano Vilas Boas, C. R.; et al. Oxidation of metal thin films by atomic oxygen: A low energy ion scattering study. J. Appl. Phys. 2019, 126 (15), 155301.
40. Stilhano Vilas Boas, C. R.; et al. Room temperature oxygen exchange and diffusion in nanometer-thick ZrO₂ and MoO₃ films. Appl. Surf. Sci. 2021, 550, 149384.
41. Sun, Z.; et al. Highly selective photocatalytic conversion of methane to liquid oxygenates over silicomolybdic-acid/TiO₂ under mild conditions. J. Mater. Chem. A 2021, 9 (3), 1713-1719.
42. Sushkevich, V. L.; et al. Selective anaerobic oxidation of methane enables direct synthesis of methanol. Science 2017, 356 (6337), 523.
43. Takata, T.; et al. Fabrication of a Core-Shell-Type Photocatalyst via Photodeposition of Group IV and V Transition Metal Oxyhydroxides: An Effective Surface Modification Method for Overall Water Splitting. J. Am. Chem. Soc. 2015, 137 (30), 9627-9634.
44. Villa, K.; et al. An insight on the role of La in mesoporous WO₃ for the photocatalytic conversion of methane into methanol. Appl. Catal., B 2016, 187, 30-36.
45. Woertink, J. S.; et al. A [Cu₂O]²⁺ core in Cu-ZSM-5, the active site in the oxidation of methane to methanol. Proc. Natl. Acad. Sci. U.S.A 2009, 106 (45), 18908.
46. Zhang, J.; et al. Quantitative Detection of OH Radicals for Investigating the Reaction Mechanism of Various Visible-Light TiO2 Photocatalysts in Aqueous Suspension. J. Phys. Chem. C 2013, 117 (3), 1383-1391.
47. Zhang, Y. et al., Boosting the catalysis of gold by O₂ activation at Au—SiO₂ interface. Nat. Commun. 11, 558 (2020).
48. Zhou, W.; et al. Highly selective aerobic oxidation of methane to methanol over gold decorated zinc oxide via photocatalysis. J. Mater. Chem. A 2020, 8 (26), 13277-13284.
49. Zhu, S.; et al. Efficient Photooxidation of Methane to Liquid Oxygenates over ZnO Nanosheets at Atmospheric Pressure and Near Room Temperature. Nano Lett. 2021, 21 (9), 4122-4128.

1. A method for oxidizing an alkane, the method comprising:
(a) contacting a composition comprising the alkane with a core-shell nanoparticle and an oxidant to produce a mixture; and
(b) irradiating the mixture to produce one or more oxidized alkane species.

2. The method of claim 1, wherein the alkane comprises a C1-C6 linear, branched, or cyclic alkane.

3. The method of claim 1, wherein the oxidant comprises O₂, H₂O₂, N₂O, or any combination thereof.

4. The method of claim 1, wherein the oxidant comprises O₂ and wherein the method is conducted with a ratio of alkane partial pressure to O₂ partial pressure of from 100:0.5 to about 2:1.

5. The method of claim 1, wherein the mixture further comprises a solvent.

6. The method of claim 5, wherein the solvent comprises water.

7. The method of claim 1, wherein a core of the core-shell nanoparticle comprises at least one semiconductor.

8. The method of claim 7, wherein the at least one semiconductor comprises TiO₂, SrTiO₃, ZnO, BiVO₄, In₂O₃, carbon nitride, or any combination thereof.

9. The method of claim 1, wherein a shell of the core-shell nanoparticle comprises at least one oxide transparent to UV or visible radiation.

10. The method of claim 9, wherein the at least one oxide comprises SiO₂.

11. The method of claim 9, wherein the shell has a thickness of from about 0.5 nm to about 20 nm.

12. The method of claim 1, wherein a shell of the core-shell nanoparticle further comprises a dopant.

13. The method of claim 12, wherein the dopant comprises gold, platinum, palladium, copper, rhenium, ruthenium, or any combination thereof.

14. The method of claim 12, wherein the dopant is present in an amount of from about 0.1 wt % to about 10 wt % relative to the total weight of the nanoparticles.

15. The method of claim 1, wherein the mixture is irradiated using light having a wavelength of from about 320 nm to about 780 nm.

16. The method of claim 1, wherein the method is carried out as a batch process or a continuous process.

17. The method of claim 1, wherein the alkane comprises methane and the one or more oxidized alkane species comprises formic acid, formaldehyde, methanol, methyl hydroperoxide, carbon dioxide, or any combination thereof.

18. The method of claim 17, wherein an amount of methanol produced is at least 4 times greater than an amount of carbon dioxide produced.

19. The method of claim 1, wherein the alkane comprises ethane and the one or more oxidized alkane species comprises acetic acid, acetaldehyde, ethanol, or any combination thereof.

20. An oxidized alkane produced by the method of claim 1.
